(19) United States
(12) Patent Application Publication
(10) Pub. No.: US 2004/0235019 A1
(43) Pub. Date: Nov. 25, 2004

(54) Diagnostics and therapeutics for the gene expression signature of PPAR-gamma receptor ligand

(75) Inventors: Justin Chapman, San Diego, CA (US); Michael North, San Diego, CA (US); Jerrold Olefsky, Solana Beach, CA (US); Dorothy Worrall, Cardiff-by-the-Sea, CA (US)

Correspondence Address: AGOURON PHARMACEUTICALS, INC., 10350 NORTH TORREY PINES ROAD, LA JOLLA, CA, 92037, US

(73) Assignees: Agouron Pharmaceuticals, Inc.; The Regents of the University of California

(21) Appl. No.: 10/791,950

(22) Filed: Mar. 3, 2004

Related U.S. Application Data
(60) Provisional application No. 60/453,122, filed on Mar. 6, 2003.

(51) Int. Cl. C12Q 1/68

(52) U.S. Cl. 435/6

(57) ABSTRACT

The invention relates to a method for identifying a therapeutic having analogous activity to a thiazolidinedione by contacting a cell containing a PPARγ receptor with a candidate therapeutic; and determining the level of a selected gene expression; to indicate that the candidate therapeutic is a therapeutic for treating a disease associated with a PPARγ receptor. The invention also relates to a composition comprising a plurality of selected genes or gene fragments, or a plurality of proteins or proteins fragments selected from proteins encoded by the selected genes thereof. The invention further relates to a method for determining whether a subject is responsive to treatment with a therapeutic having analogous activity to a thiazolidinedione.

### BACKGROUND OF THE INVENTION

This application claims benefit of priority from United States Provisional Application Number 453,122 filed on Mar. 6, 2003.

The Proliferator-Activated Receptors (PPARs) are members of the nuclear receptor superfamily that bind specific DNA response elements and in response to ligand binding, result in the activation of several genes. The PPARs, like other members of the nuclear receptor superfamily, contain a DNA-binding domain, a ligand-binding domain, and a flexible hinge connecting the two. PPARs heterodimerize with the retinoid X receptor (RXR) and bind to specific DNA response elements (PPREs). Upon ligand binding to PPAR, the receptor experiences a conformational change that results in activation of gene transcription.

PPARs include the subtypes PPARα, PPARγ, and PPARδ. Natural agonists of the three types of PPARs include fatty acids implicating them as critical regulators in metabolic pathways involving energy storage and potential targets for therapeutics against disorders such as obesity (Kliewer, et al., Recent Progress in Hormone Research, 2001, 56: 239-63). Of the three subtypes, PPARγ has been most extensively studied and is known to play an important role in the regulation of glucose and lipid homeostasis as well as in adipocyte differentiation (Willson, et al., Journal of Medicinal Chemistry, 2000, 43: 527-550). The PPARγ protein is conserved across several species including mice and humans. One of the first synthetic ligands of PPARγ identified as agonists was a class of antidiabetic compounds known as thiazolidinediones (TZDs). The relative effectiveness of individual TZDs in anti-diabetic therapy correlates with their ability to bind and activate the PPARγ receptor (Auwerx, J., Diabetologia, 1999, 42: 1033-1049). TZDs have been shown to induce gene expression in adipocytes and have been correlated with lowered glucose levels (Willson, et al.). TZDs include Rosiglitazone, Troglitazone, Pioglitazone, and MCC-555. Each of these TZDs bind preferentially to PPARγ over the other PPAR subtypes.

TZDs have been shown to reduce plasma glucose, lipid and insulin levels. Pioglitazone and rosiglitazone are Food and Drug Administration approved drugs that are currently sold for the treatment of Type II diabetes. A third TZD, troglitazone, was also FDA approved for Type II diabetes, but has been withdrawn from commercial use due to the occurrence of undesirable side effects.

The response of patients to particular TZDs are quite variable, and 20-30% of patients are classified as non-responders. In addition, the incidence of side effects can differ among subjects. Accordingly, it is highly desirable to identify compounds for treating diabetes and related conditions that are more therapeutically effective with fewer side effects. It is also highly desirable to develop more accurate methods for predicting whether a subject is likely to respond to a particular treatment as well as methods that determine the extent of a patient's response to the treatment.

### SUMMARY OF THE INVENTION

In general, the inventions are based on the identification of genes that are up- or down-regulated in cells expressing the PPARγ receptor in the presence of known PPARγ receptor ligands.

Based on these findings, in one aspect, the invention features gene and protein arrays and methods for using the same in drug discovery and pharmacogenomics.

In another aspect, the invention relates to a method for identifying a therapeutic having analogous activity to a thiazolidinedione comprising contacting a cell containing a PPARγ receptor with a candidate therapeutic; and determining the level of expression of at least one gene selected from the panel of genes in Table I and/or Table II, wherein an increase in the level of expression of at least one gene of Tables I or III and/or a decrease in the level of expression of at least one gene of Tables II or IV in the cell treated with the candidate therapeutic relative to a cell that was not treated with the candidate therapeutic indicates that the candidate therapeutic is a therapeutic for treating a disease associated with a PPARγ receptor.

In one embodiment of-this aspect of the invention, said candidate therapeutic is selected from the group consisting of: proteins, peptides, peptidomimetics, derivatives of fatty acids, and small molecules.

In another embodiment of this aspect of the invention, said disease is Type II diabetes.

In another embodiment of this aspect of the invention, said disease is obesity.

In another embodiment of this aspect of the invention, said disease is treatable by a thiazolidinedione.

In another embodiment of this aspect of the invention, said PPARγ receptor is the PPARγ1 receptor.

In another embodiment of this aspect of the invention, said PPARγ receptor is the PPARγ2 receptor.

In another embodiment of this aspect of the invention, said candidate therapeutic is in a library of compounds.

In another embodiment of this aspect of the invention, the expression level of at least three genes is detected.

In another embodiment of this aspect of the invention, the expression level of at least ten genes is detected.

In another aspect, the invention relates to a composition comprising a plurality of genes or gene fragments selected from the panel of genes in Tables I-IV.

In one embodiment of this aspect of the invention, the plurality is at least 10 genes or gene fragments.

In another embodiment of this aspect of the invention, the plurality is at least 20 genes or gene fragments.

In another embodiment of this aspect of the invention, the composition is a chip, wafer or slide.

In yet another aspect, the invention relates to a composition comprising a plurality of proteins or proteins fragments selected from proteins encoded by the panel of genes in Tables I-IV.

In one embodiment of this aspect of the invention, the plurality is at least 10 proteins or protein fragments.

In another embodiment of this aspect of the invention, the plurality is at least 20 proteins or protein fragments.

In another embodiment of this aspect of the invention, the composition is a chip, wafer or slide.

In yet another aspect, the invention relates to a method for determining whether a subject is responsive to treatment with a therapeutic having analogous activity to a thiazolidinedione, comprising determining the level of expression of a plurality of genes of Tables I or III or Tables II or IV in cells of the subject, wherein a higher level of expression of the genes of Tables I or III or a lower level of expression of the genes of Tables II or IV in the adipocytes of the subject relative to that in adipocytes of a subject that was not treated with a PPARγ ligand indicates that the subject is responsive to treatment with the PPARγ ligand.

In one embodiment of this aspect of the invention, the cells are adipocytes.

In yet another aspect, the invention relates to a method for predicting whether a subject would be responsive to treatment with a compound having analogous activity to a thiazolidinedione, comprising incubating cells of the subject with a PPARγ ligand and determining the level of expression of a plurality of genes of Tables I and/or III and/or Tables II and/or IV in the cells, wherein a higher level of expression of genes of Tables I or III or lower level of expression of genes of Tables II or IV relative to expression in cells of subjects not treated with a PPARγ ligand indicates that the subject would be responsive to treatment with the PPARγ ligand.

Other features and advantages of the instant inventions will now be described in the following Detailed Description and claims.

### BRIEF DESCRIPTION OF THE DRAWINGS

FIG. 1 shows a schematic of Venn overlap of genes up-regulated in response to PPARγ ligand treatment.

FIG. 2 shows a schematic of Venn overlap of genes down-regulated in response to PPARγ ligand treatment.

FIG. 3 shows a schematic of fold change values for selected genes from core set of genes determined by Venn overlap to be 1.5 fold up or down-regulated by 24 hour Farglitazar, Darglitazone, Rosiglitazone, Pioglitazone, and Troglitazone treatment in 3T3-L1 adipocytes.

FIG. 4 shows a schematic of Box and whisker plots of selected genes up and down-regulated by PPARγ ligand treatment.

FIG. 5 shows a schematic of Box Heat map diagram of genes found to be 1.5 fold up or down-regulated by 24 hour PPARγ ligand treatment in 3T3-L1 adipocytes. Diagram is colored by expression level and genes are grouped by biological function/pathway. Red represents up-regulated genes, black represents unchanged while green represents down-regulated genes.

FIG. 6 shows a schematic of PPARγ1 and PPARγ2 expression in murine derived 3T3L1 adipocytes before and after treatment with the PPARγ ligands pioglitazone, troglitazone, rosiglitazone, MCC-555, or the non-TZD PPARγ partial agonist/antagonist 5-chloro-1-(4-chlorobenzyl)-3-(phenylthio)-1H-indole-2-carboxylic acid (SPPARM) measured in using the Taqman assay.

FIG. 7 shows a schematic of PPARγ expression in 3T3L1 adipocytes before and after treatment with the PPARγ ligands pioglitazone, troglitazone, rosiglitazone, MCC-555, or the non-TZD PPARγ partial agonist/antagonist 5-chloro-1-(4-chlorobenzyl)-3-(phenylthio)-1H-indole-2-carboxylic acid (SPPARM) measured by Affymetrix microarray analysis.

FIG. 8 shows a schematic of the total number of up- and down-regulated genes expressed in the presence of the indicated PPARγ ligands relative to the number of genes up- and down-regulated by all ligands.

FIG. 9 shows a schematic of the total number of up- and down-regulated genes expressed in the presence of the indicated PPARγ ligands relative to the number of genes up- and down-regulated by all ligands.

FIG. 10 shows a schematic of Venn diagram overlap of genes 1.5 Fold up and down-regulated by Rosiglitazone, Pioglitazone and Troglitazone treatment in experiment 2.

FIG. 11 is a schematic of Venn diagram overlap of the core list of genes up and down-regulated by Farglitazar, Darglitazone, Rosiglitazone, Pioglitazone, and Troglitazone treatment, and the independently derived list of genes 1.5 fold up or down-regulated by Rosiglitazone, Pioglitazone and Troglitazone treatment in experiment 2.

### DETAILED DESCRIPTION OF THE INVENTION

1. General

In general, the present inventions are based on the identification of genes or gene products that were found to be either up-regulated (Tables I and III) or down-regulated (Tables II and IV) in adipose cells expressing the PPARγ receptor in the presence of known ligands of the PPARγ receptor. As described further herein, these genes or gene panels are useful for identifying therapeutics for treating PPARγ-associated diseases and in pharmacogenomic applications.

2. Definitions

For convenience, before further description of the present invention, certain terms employed in the specification, examples and appended claims are defined here.

The singular forms “a”, “an”, and “the” include plural references unless the context clearly dictates otherwise.

An “address” on an array, e.g., a microarray, refers to a location at which an element, e.g., an oligonucleotide, is attached to the solid surface of the array. As used herein, a nucleic acid or other molecule attached to an array, is referred to as a “probe” or “capture probe.” When an array contains several probes corresponding to one gene, these probes are referred to as “gene-probe set.” A gene-probe set may consist of, e.g., 2 to 10 probes, preferably from 2 to 5 probes and most preferably about 5 probes.

“Agonist” refers to an agent that mimics or up-regulates (e.g., potentiates or supplements) the bioactivity of a protein, e.g., polypeptide X. An agonist may be a wild-type protein or derivative thereof having at least one bioactivity of the wild-type protein. An agonist may also be a compound that up-regulates expression of a gene or which increases at least one bioactivity of a protein. An agonist may also be a compound which increases the interaction of a polypeptide with another molecule, e.g., a target peptide or nucleic acid.

“Allele”, which is used interchangeably herein with “allelic variant”, refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene may differ from each other in a single nucleotide, or several nucleotides, and may include substitutions, deletions, and insertions of nucleotides. An allele of a gene may also be a form of a gene containing a mutation.

“Amplification,” refers to the production of additional copies of a nucleic acid sequence. Amplification is generally carried out using polymerase chain reaction (PCR) technologies well known in the art. (Dieffenbach, C. W. and G. S. Dveksler, (1995) PCR Primer: a Laboratory Manual, Cold Spring Harbor Press, Plainview, N.Y.)

“Antagonist” refers to an agent that down-regulates (e.g., suppresses or inhibits) at least one bioactivity of a protein. An antagonist may be a compound which inhibits or decreases the interaction between a protein and another molecule, e.g., a target peptide or enzyme substrate. An antagonist may also be a compound that down-regulates expression of a gene or which reduces the amount of expressed protein present.

“Antibody” is intended to include whole antibodies of any isotype (e.g., IgG, IgA, IgM, IgE, etc.), and includes fragments thereof which are also specifically reactive with a vertebrate, e.g., mammalian, protein. Antibodies may be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)₂, Fab′, Fv, and single chain antibodies (scFv) containing a V[L] and/or V[H] domain joined by a peptide linker. The scFv's may be covalently or non-covalently linked to form antibodies having two or more binding sites. The subject invention includes polyclonal, monoclonal, humanized, or other purified preparations of antibodies and recombinant antibodies.

“Antisense” nucleic acid refers to oligonucleotides which specifically hybridize (e.g., bind) under cellular conditions with a gene sequence, such as at the cellular mRNA and/or genomic DNA level, so as to inhibit expression of that gene, e.g., by inhibiting transcription and/or translation. The binding may be by conventional base pair complementarily, or, for example, in the case of binding to DNA duplexes, through specific interactions in the major groove of the double helix.

“Array” or “matrix” refer to an arrangement of addressable locations or “addresses” on a device. The locations may be arranged in two dimensional arrays, three dimensional arrays, or other matrix formats. The number of locations may range from several to at least hundreds of thousands. Most importantly, each location represents a totally independent reaction site. A “nucleic acid array” refers to an array containing nucleic acid probes, such as oligonucleotides or larger portions of genes. The nucleic acid on the array is preferably single stranded. Arrays wherein the probes are oligonucleotides are referred to as “oligonucelotide arrays” or “oligonucleotide chips” or “gene chips”. A “microarray”, also referred to as a “chip”, “biochip”, or “biological chip”, is an array of regions having a suitable density of discrete regions, e.g., of at least 100/cm², and preferably at least about 1000/cm². The regions in a microarray have dimensions, e.g. diameters, preferably in the range of between about 10-250 microns, and are separated from other regions in the array by the same distance.

“Biological activity” or “bioactivity” or “activity” or “biological function”, which are used interchangeably, refer to an effector or antigenic function that is directly or indirectly performed by a polypeptide (whether in its native or denatured conformation), or by any subsequence thereof. Biological activities include binding to polypeptides, binding to other proteins or molecules, activity as a DNA binding protein, as a transcription regulator, ability to bind damaged DNA, etc. A bioactivity may be modulated by directly affecting the subject polypeptide. Alternatively, a bioactivity may be altered by modulating the level of the polypeptide, such as by modulating expression of the corresponding gene.

“Biological sample” or “sample”, refers to a sample obtained from an organism or from components (e.g., cells) of an organism. The sample may be of any biological tissue or fluid. Frequently the sample will be a “clinical sample” which is a sample derived from a patient. Such samples include, but are not limited to, sputum, blood, blood cells (e.g., white cells), tissue or fine needle biopsy samples, urine, peritoneal fluid, and pleural fluid, or cells therefrom. Biological samples may also include sections of tissues such as frozen sections taken for histological purposes.

“Biomarker” refers to a biological molecule whose presence, concentration, activity, or post-translationally-modified state may be detected and correlated with the activity of a protein of interest.

A “combinatorial library” or “library” is a plurality of compounds, which may be termed “members,” synthesized or otherwise prepared from one or more starting materials by employing either the same or different reactants or reaction conditions at each reaction in the library. In general, the members of any library show at least some structural diversity, which often results in chemical diversity. A library may have anywhere from two different members to about 10⁸ members or more. In certain embodiments, libraries of the present invention have more than about 12, 50 and 90 members. In certain embodiments of the present invention, the starting materials and certain of the reactants are the same, and chemical diversity in such libraries is achieved by varying at least one of the reactants or reaction conditions during the preparation of the library. Combinatorial libraries of the present invention may be prepared in solution or on the solid phase.

“Complementary” or “complementarity”, refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between the single stranded molecules. The degree of complementarity between nucleic acid strands has significant effects on the efficiency and strength of hybridization between nucleic acid strands.

A “delivery complex” refers to a targeting means (e.g. a molecule that results in higher affinity binding of a gene, protein, polypeptide or peptide to a target cell surface and/or increased cellular or nuclear uptake by a target cell). Examples of targeting means include: sterols (e.g. cholesterol), lipids (e.g. a cationic lipid, virosome or liposome), viruses (e.g. adenovirus, adeno-associated virus, and retrovirus) or target cell specific binding agents (e.g. ligands recognized by target cell specific receptors). Preferred complexes are sufficiently stable in vivo to prevent significant uncoupling prior to internalization by the target cell. However, the complex is cleavable under appropriate conditions within the cell so that the gene, protein, polypeptide or peptide is released in a functional form.

“Derived from” as that phrase is used herein indicates a peptide or nucleotide sequence selected from within a given sequence. A peptide or nucleotide sequence derived from a named sequence may contain a small number of modifications relative to the parent sequence, in most cases representing deletion, replacement or insertion of less than about 15%, preferably less than about 10%, and in many cases less than about 5%, of amino acid residues or base pairs present in the parent sequence. In the case of DNAs, one DNA molecule is also considered to be derived from another if the two are capable of selectively hybridizing to one another.

“Derivative” refers to the chemical modification of a polypeptide sequence, a polynucleotide sequence or a class of small molecules, such as fafty acids. Chemical modifications of a polynucleotide sequence may include, for example, replacement of hydrogen by an alkyl, acyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

“Differentiation” refers to the process by which a cell becomes specialized for a specific structure or function by selective gene expression of some genes and selective repression of others.

“Differential expression” refers to both quantitative as well as qualitative differences in a gene's temporal and/or tissue expression patterns. Differentially expressed genes may represent “target genes.”

“Differential gene expression pattern” between cell A and cell B refers to a pattern reflecting the differences in gene expression between cell A and cell B. A differential gene expression pattern may also be obtained between a cell at one time point and a cell at another time point, or between a cell incubated or contacted with a compound and a cell that was not incubated or contacted with the compound.

“Disease associated with PPARγ” or “a disease associated with a PPARγ receptor” includes diseases treatable with TZDs, or other ligands of PPARγ, such as but not limited to Type II diabetes and obesity. Diseases related to PPARγ expression and/or activity would also be considered as associated with the PPARγ receptor, such as obesity or other disorders expected to be affected by alterations in PPARγ's role in activating adipocyte differentiation.

“Equivalent” refers to nucleotide sequences encoding functionally equivalent polypeptides. Equivalent nucleotide sequences will include sequences that differ by one or more nucleotide substitutions, additions or deletions, such as allelic variants; and will, therefore, include sequences that differ from the nucleotide sequence of the nucleic acids referred to in the Tables due to the degeneracy of the genetic code.

“Expression profile,” which is used interchangeably herein with “gene expression profile,” “expression signature” and “finger print” of a cell, refers to a set of values representing mRNA levels of a plurality of genes in a cell. An expression profile preferably comprises values representing expression levels of at least about 10 genes. Expression profiles preferably comprise an mRNA level of a gene which is expressed at similar levels in multiple cells and conditions, e.g., GAPDH. For example, an expression profile of a diseased cell refers to a set of values representing mRNA levels of 10 or more genes in a diseased cell.

The “level of expression of a gene in a cell” or “gene expression level” refers to the level of mRNA, as well as pre-mRNA nascent transcript(s), transcript processing intermediates, mature mRNA(s) and degradation products, encoded by the gene in the cell.

“Gene” or “recombinant gene” refer to a nucleic acid molecule comprising an open reading frame and including at least one exon and (optionally) an intron sequence. “Intron” refers to a DNA sequence present in a given gene which is spliced out during mRNA maturation.

“Gene construct” refers to a vector, plasmid, viral genome or the like which includes a “coding sequence” for a polypeptide or which is otherwise transcribable to a biologically active RNA (e.g., antisense, decoy, ribozyme, etc), may transfect cells, in certain embodiments mammalian cells, and may cause expression of the coding sequence in cells transfected with the construct. The gene construct may include one or more regulatory elements operably linked to the coding sequence, as well as intronic sequences, poly adenylation sites, origins of replication, marker genes, etc.

“Homology” or alternatively “identity” refers to sequence similarity between two peptides or between two nucleic acid molecules. Homology may be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The term “percent identical” refers to sequence identity between two amino acid sequences or between two nucleotide sequences. Identity may each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules may be referred to as homologous (similar) at that position. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.). ENTREZ is available through the National Center for Biotechnology Information, National Library of Medicine, National Institutes of Health, Bethesda, Md. In one embodiment, the percent identity of two sequences may be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis, 1996, ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol., 1997, 70: 173-187. Also, the GAP program using the Needleman and Wunsch alignment method may be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences may be used to search both protein and DNA databases. Databases with individual sequences are described in Methods in Enzymology, ed. Doolittle, supra. Databases include Genbank, EMBL, and DNA Database of Japan (DDBJ).

“Host cell” refers to a cell transduced with a specified transfer vector. The cell is optionally selected from in vitro cells such as those derived from cell culture, ex vivo cells, such as those derived from an organism, and in vivo cells, such as those in an organism.

“Recombinant host cells” refers to cells which have been transformed or transfected with vectors constructed using recombinant DNA techniques. “Host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.”

“Hybridization” refers to any process by which a strand of nucleic acid binds with a complementary strand through base pairing.

“Specific hybridization” of a probe to a target site of a template nucleic acid refers to hybridization of the probe predominantly to the target, such that the hybridization signal may be clearly interpreted. As further described herein, such conditions resulting in specific hybridization vary depending on the length of the region of homology, the GC content of the region, the melting temperature “Tm” of the hybrid. Hybridization conditions will thus vary in the salt content, acidity, and temperature of the hybridization solution and the washes.

“Interact” is meant to include detectable interactions between molecules, such as may be detected using, for example, a hybridization assay. Interact also includes “binding” interactions between molecules. Interactions may be, for example, protein-protein, protein-nucleic acid, protein-small molecule or small molecule-nucleic acid in nature.

“Isolated”, with respect to nucleic acids, such as DNA or RNA, refers to molecules separated from other DNAs, or RNAs, respectively, that are present in the natural source of the macromolecule. Isolated also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. “Isolated” also refers to polypeptides which are isolated from other cellular proteins and is meant to encompass both purified and recombinant polypeptides.

“Label” and “detectable label” refer to a molecule capable of detection, including, but not limited to, radioactive isotopes, fluorophores, chemiluminescent moieties, enzymes, enzyme substrates, enzyme cofactors, enzyme inhibitors, dyes, metal ions, ligands (e.g., biotin or haptens) and the like. “Fluorophore” refers to a substance or a portion thereof which is capable of exhibiting fluorescence in the detectable range. Particular examples of labels which may be used under the invention include fluorescein, rhodamine, dansyl, umbelliferone, Texas red, luminol, NADPH, alpha-beta-galactosidase and horseradish peroxidase.

A “molecular target” or “target” refers to a molecular structure that is a gene or derived from a gene that has been identified in a sample or diseased cell using the methods of the invention as exhibiting differential expression relative to the gene in a control or normal cell of interest. Exemplary targets as such are polypeptides, hormones, receptors, dsDNA fragments, carbohydrates or enzymes. Such targets also may be referred to as “target genes”, “target peptides”, “target proteins”, and the like.

“Modulation” refers to up regulation (i.e., activation or stimulation), down regulation (i.e., inhibition or suppression) of a response, or the two in combination or apart.

“Nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

“Nucleic acid corresponding to a gene” refers to a nucleic acid that may be used for detecting the gene, e.g., a nucleic acid which is capable of hybridizing specifically to the gene.

“Nucleic acid sample derived from RNA” refers to one or more nucleic acid molecule, e.g., RNA or DNA, that was synthesized from the RNA, and includes DNA resulting from methods using PCR, e.g., RT-PCR.

“Panel” as used herein refers to a group of genes and/or their encoded proteins identified via a gene expression profile as being differentially expressed upon treatment with a PPARγ ligand.

A “patient”, “subject” or “host” to be treated by the subject method may mean either a human or non-human animal.

“Peptidomimetic” refers to a compound containing peptide-like structural elements that is capable of mimicking the biological action (s) of a natural parent polypeptide.

“Percent identical” refers to sequence identity between two amino acid sequences or between two nucleotide sequences. Identity may each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules may be referred to as homologous (similar) at that position. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and may be used with, e.g., default settings. ENTREZ is available through the National Center for Biotechnology Information, National Library of Medicine, National Institutes of Health, Bethesda, Md. In one embodiment, the percent identity of two sequences may be determined by the GCG program with a gap weight of 1, e.g., each amino acid gap is weighted as if it were a single amino acid or nucleotide mismatch between the two sequences. Other techniques for alignment are described in Methods in Enzymology, vol. 266: Computer Methods for Macromolecular Sequence Analysis, 1996, ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol., 1997, 70: 173-187. Also, the GAP program using the Needleman and Wunsch alignment method may be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences may be used to search both protein and DNA databases. Databases with individual sequences are described in Methods in Enzymology, ed. Doolittle, supra. Databases include Genbank, EMBL, and DNA Database of Japan (DDBJ).

“Perfectly matched” in reference to a duplex means that the poly- or oligonucleotide strands making up the duplex form a double stranded structure with one other such that every nucleotide in each strand undergoes Watson-Crick basepairing with a nucleotide in the other strand. The term also comprehends the pairing of nucleoside analogs, such as deoxyinosine, nucleosides with 2-aminopurine bases, and the like, that may be employed. A mismatch in a duplex between a target polynucleotide and an oligonucleotide or olynucleotide means that a pair of nucleotides in the duplex fails to undergo Watson-Crick bonding. In reference to a triplex, the term means that the triplex consists of a perfectly matched duplex and a third strand in which every nucleotide undergoes Hoogsteen or reverse Hoogsteen association with a basepair of the perfectly matched duplex.

“Pharmaceutically-acceptable salts” refers to the relatively non-toxic, inorganic and organic acid addition salts of compounds.

“Pharmaceutically acceptable carrier” refers to a pharmaceutically-acceptable material, composition or vehicle, such as a liquid or solid filler, diluent, excipient, solvent or encapsulating material, involved in carrying or transporting any supplement or composition, or component thereof, from one organ, or portion of the body, to another organ, or portion of the body. Each carrier must be “acceptable” in the sense of being compatible with the other ingredients of the supplement and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The “profile” of a cell's biological state refers to the levels of various constituents of a cell that are known to change in response to drug treatments and other perturbations of the cell's biological state. Constituents of a cell include levels of RNA, levels of protein abundances, or protein activity levels.

An expression profile in one cell is “similar” to an expression profile in another cell when the level of expression of the genes in the two profiles are sufficiently similar that the similarity is indicative of a common characteristic, e.g., being one and the same type of cell. Accordingly, the expression profiles of a first cell and a second cell are similar when at least 75% of the genes that are expressed in the first cell are expressed in the second cell at a level that is within a factor of two relative to the first cell.

“Prophylactic” or “therapeutic” treatment refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

“Protein”, “polypeptide” and “peptide” are used interchangeably herein when referring to a gene product, e.g., as may be encoded by a coding sequence. By “gene product” it is meant a molecule that is produced as a result of transcription of a gene. Gene products include RNA molecules transcribed from a gene, as well as proteins translated from such transcripts.

“Recombinant protein”, “heterologous protein” and “exogenous protein” are used interchangeably to refer to a polypeptide which is produced by recombinant DNA techniques, wherein generally, DNA encoding the polypeptide is inserted into a suitable expression vector which is in turn used to transform a host cell to produce the heterologous protein. That is, the polypeptide is expressed from a heterologous nucleic acid.

“Small molecule” refers to a composition, which has a molecular weight of less than about 1000 kDa. Small molecules may be nucleic acids, peptides, polypeptides, peptidomimetics, carbohydrates, lipids or other organic (carbon-containing) or inorganic molecules. As those skilled in the art will appreciate, based on the present description, extensive libraries of chemical and/or biological mixtures, often fungal, bacterial, or algal extracts, may be screened with any of the assays of the invention to identify compounds that modulate a bioactivity.

“Systemic administration,” “administered systemically,” “peripheral administration” and “administered peripherally” refer to the administration of a subject supplement, composition, therapeutic or other material other than directly into the central nervous system, such that it enters the patient's system and, thus, is subject to metabolism and other like processes, for example, subcutaneous administration.

“Therapeutic agent” or “therapeutic” refers to an agent capable of having a desired biological effect on a host. Chemotherapeutic and genotoxic agents are examples of therapeutic agents that are generally known to be chemical in origin, as opposed to biological, or cause a therapeutic effect by a particular mechanism of action, respectively. Examples of therapeutic agents of biological origin include growth factors, hormones, and cytokines. A variety of therapeutic agents are known in the art and may be identified by their effects. Certain therapeutic agents are capable of regulating cell proliferation and differentiation. Examples include chemotherapeutic nucleotides, drugs, hormones, non-specific (non-antibody) proteins, oligonucleotides (e.g., antisense oligonucleotides that bind to a target nucleic acid sequence (e.g., mRNA sequence)), peptides, and peptidomimetics.

“Therapeutic effect” refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable, benefit/risk ratio applicable to such treatment.

“Treating” a disease in a subject or “treating” a subject having a disease refers to subjecting the subject to a pharmaceutical treatment, e.g., the administration of a drug, such that at least one symptom of the disease is decreased or prevented.

“Variant,” when used in the context of a polynucleotide sequence, may encompass a polynucleotide sequence related to that of gene X or the coding sequence thereof. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one base. The presence of SNPs may be indicative of, for example, a certain population, a disease state, or a propensity for a disease state.

A “variant” of polypeptide X refers to a polypeptide having the amino acid sequence of peptide X in which is altered in one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological or immunological activity may be found using computer programs well known in the art, for example, LASERGENE software (DNASTAR).

“Vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops, which, in their vector form are not bound to the chromosome. In the present specification, “plasmid” and “vector” are used interchangeably as the plasmid is the most commonly used form of vector. However, as will be appreciated by those skilled in the art, the invention is intended to include such other forms of expression vectors which serve equivalent functions and which become known in the art subsequently hereto.

3. Methods for Identifying Novel Therapeutics for Treating a Disease Associated with a PPARγ Receptor

The present invention provides panels of known genes or gene products that were discovered to exhibit similar changes in expression patterns in adipose cells as a function of culturing the adipose cells expressing the PPARγ receptor in the presence of known ligands. The genes and/or encoded gene products that comprise one panel are selected from the group of genes listed in Tables I and III and are up-regulated in the presence of all PPARγ ligands tested. The genes and/or encoded gene products that comprise another panel are selected from the group of genes listed in Tables II and IV and are down-regulated in the presence of all PPARγ ligands tested. These genes which are either up-regulated or down-regulated in the presence of PPARγ ligands, and their gene products are contemplated as probes for diagnostics and targets for drug discovery.

The PPARγ receptor modulates the expression of a number of genes in response to binding its ligand. One of the diseases treated by ligands of the PPARγ receptor is Type II diabetes, which affects a major portion of the population. A series of anti-diabetic drugs known as thiazolidinediones (TZDs) are available for treatment of Type II diabetes. However the side effects and efficacy of the drugs varies between individuals. Modified versions of the currently known TZDs could be screened as candidate therapeutics by the methods of this invention. PPARγ is known to play a critical role in the activation of adipocyte differentiation and candidate therapeutics could be directed towards diseases treatable by inhibiting adipocyte proliferation, such as, but not limited to, obesity.

As described above, the panels of genes which are either up-regulated or down-regulated as a function of treatment with multiple PPARγ ligands are contemplated for use in the present invention as targets in drug design and discovery. In one embodiment of the invention, groups of genes selected from the panels of the present invention, and/or their encoded gene products, comprise the “targets” for these methods. In some embodiments, candidate therapeutic agents, or “therapeutics” are evaluated for their ability to up-regulate or down-regulate a group of genes selected from the panels of the present invention, and/or their encoded gene products. The candidate therapeutics may be selected from the following classes of compounds: proteins, peptides, peptidomimetics, derivatives of fatty acids, or small molecules. The candidate therapeutics may also be selected from the following classes of compounds: antisense nucleic acids, small molecules, polypeptides, proteins including antibodies, peptidomimetics, derivatives of fatty acids, or nucleic acid analogs. In some embodiments, the candidate therapeutics are selected from a library of compounds. These libraries may be generated using combinatorial synthetic methods.

The present invention provides methods for evaluating candidate therapeutic agents for their ability to increase the expression of a number of genes selected from Table I by contacting cells expressing the PPARγ receptor with molecules to be tested as potential therapeutic agents. The present invention further provides methods for evaluating candidate therapeutic agents of the present invention for their ability to decrease the expression of a number of genes selected from Table II by contacting cells expressing the PPARγ receptor with molecules to be tested as potential therapeutic agents. Alternatively, candidate therapeutic agents may be evaluated for their ability to stimulate the activity of a set of proteins encoded by the genes selected from Table I by contacting cells expressing the PPARγ receptor with molecules to be tested as potential therapeutic agents. Similarly, candidate therapeutic agents may be evaluated for their ability to inhibit the activity of a set of proteins encoded by the genes selected from Table II by contacting cells expressing the PPARγ receptor with molecules to be tested as potential therapeutic agents. Furthermore, candidate therapeutic agents may be evaluated for their ability to increase the levels of expression of a set of proteins encoded by the genes selected from Tables I and III by contacting cells expressing the PPARγ receptor with molecules to be tested as potential therapeutic agents. Similarly, candidate therapeutic agents may be evaluated for their ability to decrease the levels of expression of a set of proteins encoded by the genes selected from Tables II and IV by contacting cells expressing the PPARγ receptor with molecules to be tested as potential therapeutic agents.

Those skilled in the art will appreciate from the present description that candidate therapeutics may be identified based on their ability to bind one or more genes or the products of one or more genes identified by the present invention as up-regulated or down-regulated by ligands of the PPARγ receptor. In one embodiment, the ability of a candidate therapeutic to bind the PPARγ receptor may be evaluated by an in vivo assay using cells that express the PPARγ receptor. In another embodiment, the ability of a candidate therapeutic to bind one or more genes or the products of one or more genes identified by the present invention as up-regulated or down-regulated by ligands of the PPARγ receptor may be evaluated by an in vivo assay using cells that express the PPARγ receptor.

In further embodiments of the present invention, the ability of a candidate therapeutic to bind the PPARγ receptor may be evaluated by an in vitro assay with a sufficiently purified PPARγ receptor. In certain embodiments of the present invention, the ability of a candidate therapeutic to bind the genes modulated by ligands of the PPARγ receptor may be evaluated by an in vitro assay with a sufficiently purified mixture of the essential components of such an assay. In certain other embodiments of the present invention, the ability of a candidate therapeutic to bind the products of genes modulated by ligands of the PPARγ receptor may be evaluated by an in vitro assay with a sufficiently purified mixture of the essential components of such an assay.

A person of skill in the art will recognize that in certain screening assays, it will be sufficient to assess the level of expression of a single gene and that in other assays, the expression of two or more genes is preferred, whereas still in others, the expression of essentially all of the genes up-regulated or down-regulated by ligands of the PPARγ receptor is preferably assessed. Likewise, it will be sufficient to assess the activity of a single protein in some screening assays, whereas in others, the activities of multiple proteins may be assessed. Examples of assays contemplated for use in order to screen for ligands of the PPARγ receptor include, but are not limited to, the direct binding assay, the competitive binding assay, cell proliferation assay etc. Examples of assays contemplated for use in order to assess the expression levels of RNA, levels of proteins or activity of proteins include, but are not limited to, reverse transcription assays, polymerase chain reaction (PCR) assays, Real Time-PCR assays, Northern blot assays, immunoprecipitation assays, Western blot assays, etc. Such assays are well known to one of skill in the art and, based on the present description, may be adapted to the methods of the present invention with no more than routine experimentation as described below in Sections 5 and 6.

4. Pharmacogenomic Methods.

The present invention provides methods for determining the efficacy of a candidate therapeutic as a drug for a disease associated with a PPARγ receptor. In one embodiment, a method for determining efficacy may comprise the steps of a) contacting a candidate therapeutic to an adipose cell of a subject; and b) determining the ability of said candidate therapeutic to produce an expression profile indicative of the expression signature of ligands of the PPARγ receptor of the invention.

Additionally, candidate therapeutics can be screened for efficacy by monitoring for the increased expression level of one or more genes from Tables I and III identified as up-regulated by ligands of the PPARγ receptor after incubating an adipose cell of a subject having a disease associated with the PPARγ receptor, such as Type 11 diabetes, with the test compound. In a similar embodiment, candidate therapeutics can be screened for efficacy by monitoring for the decreased expression level of one or more genes from Tables II and IV identified as down-regulated by ligands of the PPARγ receptor after incubating an adipose cell of a subject having a disease associated with the PPARγ receptor, such as Type II diabetes, with the test compound.

Test compounds will be screened for those which alter the level of expression of genes characteristic of the ligands of the PPARγ receptor, so as to bring them to a level that is similar to that in a cell exposed to the known ligands of the PPARγ receptor of the invention. Such compounds, i.e., compounds which are capable of producing the same expression profile as the known ligands of the PPARγ receptor, are candidate therapeutics.

The efficacy of the compounds may then be tested in additional in vitro and in vivo assays in adipose cells extracted from a mammalian subject. A test compound may be administered to a test animal and the gene expression profile monitored. The increased expression of one or more genes from Tables I and III may be measured before and after administration of the test compound to the mammal. Similarly, the decreased expression of one or more genes from Tables II and IV may also be measured before and after administration of the test compound to the mammal. Increased or decreased expression of one or more of these genes from either Tables I and III or Tables II and IV respectively is indicative of the efficiency of the compound for treating a disease associated with the PPARγ receptor in the mammal.

In another embodiment of the invention, a drug is developed by rational drug design, i.e., it is designed or identified based on information stored in computer readable form and analyzed by algorithms. More and more databases of expression profiles are currently being established, numerous ones being publicly available. By screening such databases for the description of drugs affecting the expression of at least some of the genes from Tables I-IV in a manner similar to the change in gene expression profile described by this invention could lead to the identification of compounds with are candidate therapeutics. Derivatives and analogues of such compounds may then be synthesized to optimize the activity of the compound, and tested and optimized as described above.

Compounds identified by the methods described above are within the scope of the invention. Compositions comprising such compounds, in particular, compositions comprising a pharmaceutically efficient amount of the drug in a pharmaceutically acceptable carrier are also provided. Certain compositions comprise one or more active compound for treating a disease associated with a PPARγ receptor such as, but not limited to, Type II diabetes or obesity.

The invention also provides methods for designing therapeutics for treating diseases associated with the PPARγ receptor. A compound for treating Type II diabetes may be derivatized and tested as further described herein.

Methods for monitoring the expression of genes, gene products, or protein activity are further discussed below in Section 5 and 6.

5. Probes

The present invention also provides probes derived from the genes or encoded proteins listed in Tables I and II. These probes are contemplated for use in diagnostic applications as discussed herein. The probes may also be prepared as panels comprising at least 1, preferably at least 3, at least 5, at least 10 or at least 20 genes from Tables I-IV. The panels may comprise probes corresponding to each gene listed in Tables I and II, or subsets of those genes in Tables I-IV which are up-regulated or down-regulated by PPARγ ligands.

In one embodiment of the present invention, the panel is arranged as a microarray. There may be one or more than one probe corresponding to each gene on a microarray. For example, a microarray may contain from 2 to 20 probes corresponding to one gene and preferably about 5 to 10. The probes may correspond to the full length RNA sequence or complements thereof of genes from Tables I-IV, or they may correspond to a portion thereof, which portion is of sufficient length for permitting specific hybridization. Such probes may comprise from about 50 nucleotides to about 100, 200, 500, or 1000 nucleotides or more than 1000 nucleotides. As further described herein, microarrays may contain oligonucleotide probes, consisting of about 10 to 50 nucleotides, preferably about 15 to 30 nucleotides and even more preferably 20-25 nucleotides. The probes are preferably single stranded. The probe will have sufficient complementarity to its target to provide for the desired level of sequence specific hybridization (see below).

Typically, the arrays used in the present invention will have a site density of greater than 100 different probes per cm² although any suitable site density is included in the present invention. Preferably, the arrays will have a site density of greater than 500/cm², more preferably greater than about 1000/cm², and most preferably, greater than about 10,000/cm². Preferably, the arrays will have more than 100 different probes on a single substrate, more preferably greater than about 1000 different probes still more preferably, greater than about 10,000 different probes and most preferably, greater than 100,000 different probes on a single substrate.

Microarrays maybe prepared by methods known in the art, as described below, or they may be custom made by companies, e.g., Affymetrix (Santa Clara, Calif.).

Generally, two types of microarrays maybe used. These two types are referred to as “synthesis” and “delivery.” In the synthesis type, a microarray is prepared in a step-wise fashion by the in situ synthesis of nucleic acids from nucleotides. With each round of synthesis, nucleotides are added to growing chains until the desired length is achieved. In the delivery type of microarray, pre-prepared nucleic acids are deposited onto known locations using a variety of delivery technologies. Numerous articles describe the different microarray technologies, e.g., Shena, et al., Tibtech, 1998, 16: 301; Duggan, et al., Nat. Genet., 1999, 21:10; Bowtell, et al., Nat. Genet., 1999, 21:25.

One novel synthesis technology is that developed by Affymetrix (Santa Clara, Calif.), which combines photolithography technology with DNA synthetic chemistry to enable high density oligonucleotide microarray manufacture. Such chips contain up to 400,000 groups of oligonucleotides in an area of about 1.6 cm². Oligonucleotides are anchored at the 3′ end thereby maximizing the availability of single-stranded nucleic acid for hybridization. Generally such chips, referred to as “GeneChips®” contain several oligonucleotides of a particular gene, e.g., between 15-20, such as 16 oligonucleotides. Since Affymetrix (Santa Clara, Calif.) sells custom made microarrays, microarrays containing genes from Tables I and II maybe ordered for purchase from Affymetrix (Santa Clara, Calif.).

Microarrays may also be prepared by mechanical microspotting, e.g., those commercialized at Synteni (Fremont, Calif.). According to these methods, small quantities of nucleic acids are printed onto solid surfaces. Microspotted arrays prepared at Synteni contain as many as 10,000 groups of cDNA in an area of about 3.6 cm².

A third group of microarray technologies consist of the “drop-on-demand” delivery approaches, the most advanced of which are the ink-jetting technologies, which utilize piezoelectric and other forms of propulsion to transfer nucleic acids from miniature nozzles to solid surfaces. Inkjet technologies is developed at several centers including Incyte Pharmaceuticals (Palo Alto, Calif.) and Protogene (Palo Alto, Calif.). This technology results in a density of 10,000 spots per cm². See also, Hughes, et al., Nat. Biotechn., 2001, 19:342.

Arrays preferably include control and reference nucleic acids. Control nucleic acids are nucleic acids which serve to indicate that the hybridization was effective. For example, all Affymetrix (Santa Clara, Calif.) expression arrays contain sets of probes for several prokaryotic genes, e.g., bioB, bioC and bioD from biotin synthesis of E. coli and cre from P1 bacteriophage. Hybridization to these arrays is conducted in the presence of a mixture of these genes or portions thereof, such as the mix provided by Affymetrix (Santa Clara, Calif.) to that effect (Part Number 900299), to thereby confirm that the hybridization was effective. Control nucleic acids included with the target nucleic acids may also be mRNA synthesized from cDNA clones by in vitro transcription. Other control genes that may be included in arrays are polyA controls, such as dap, lys, phe, thr, and trp (which are included on Affymetrix GeneChips®)

Reference nucleic acids allow the normalization of results from one experiment to another, and the comparison of multiple experiments on a quantitative level. Exemplary reference nucleic acids include housekeeping genes of known expression levels, e.g., GAPDH, hexokinase and actin.

Mismatch controls may also be provided for the probes to the target genes, for expression level controls, specificity, or for normalization controls. Mismatch controls are oligonucleotide probes or other nucleic acid probes identical to their corresponding test or control probes except for the presence of one or more mismatched bases.

Arrays may also contain probes that hybridize to more than one allele of a gene. For example the array may contain one probe that recognizes allele 1 and another probe that recognizes allele 2 of a particular gene.

Microarrays maybe prepared as follows. In one embodiment, an array of oligonucleotides is synthesized on a solid support. Exemplary solid supports include glass, plastics, polymers, metals, metalloids, ceramics, organics, etc. Using chip masking technologies and photoprotective chemistry it is possible to generate ordered arrays of nucleic acid probes. These arrays, which are known, e.g., as “DNA chips,” or as very large scale immobilized polymer arrays (“VLSPS™” arrays) may include millions of defined probe regions on a substrate having an area of about 1 cm² to several cm², thereby incorporating sets of from a few to millions of probes (see, e.g., U.S. Pat. No. 5,631,734).

The construction of solid phase nucleic acid arrays to detect target nucleic acids is well described in the literature. See, Fodor, et al., Science, 1991, 251: 767-777; Sheldon, et al., Clinical Chemistry, 1993, 39(4): 718-719; Kozal, et al., Nature Medicine, 1996, 2(7): 753-759 and Hubbell, U.S. Pat. No. 5,571,639; Pinkel, et al., PCT/US95/16155 (WO 96/17958); U.S. Pat. Nos. 5,677,195; 5,624,711; 5,599,695; 5,451,683; 5,424,186; 5,412,087; 5,384,261; 5,252,743 and 5,143,854; PCT Patent Publication Nos. 92/10092 and 93/09668; and PCT WO 97/10365. In brief, a combinatorial strategy allows for the synthesis of arrays containing a large number of probes using a minimal number of synthetic steps. For instance, it is possible to synthesize and attach all possible DNA 8 mer oligonucleotides (48, or 65,536 possible combinations) using only 32 chemical synthetic steps. In general, VLSIPS™ procedures provide a method of producing 4n different oligonucleotide probes on an array using only 4n synthetic steps (see, e.g., U.S. Pat. No. 5,631,734 5,143,854 and PCT Patent Publication Nos. WO 90/15070; WO 95/11995 and WO 92/10092).

Light-directed combinatorial synthesis of oligonucleotide arrays on a glass surface maybe performed with automated phosphoramidite chemistry and chip masking techniques similar to photoresist technologies in the computer chip industry. Typically, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′-photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface.

Algorithms for design of masks to reduce the number of synthesis cycles are described by Hubbel, et al., U.S. Pat. No. 5,571,639 and U.S. Pat. No. 5,593,839. A computer system may be used to select nucleic acid probes on the substrate and design the layout of the array as described in U.S. Pat. No. 5,571,639.

Another method for synthesizing high density arrays is described in U.S. Pat. No. 6,083,697. This method utilizes a novel chemical amplification process using a catalyst system which is initiated by radiation to assist in the synthesis the polymer sequences. Methods of the present invention include the use of photosensitive compounds which act as catalysts to chemically alter the synthesis intermediates in a manner to promote formation of polymer sequences. Such photosensitive compounds include what are generally referred to as radiation-activated catalysts (RACs), and more specifically photo activated catalysts (PACs). The RACs may by themselves chemically alter the synthesis intermediate or they may activate an autocatalytic compound which chemically alters the synthesis intermediate in a manner to allow the synthesis intermediate to chemically combine with a later added synthesis intermediate or other compound.

Arrays may also be synthesized in a combinatorial fashion by delivering monomers to cells of a support by mechanically constrained flowpaths. See Winkler, et al., EP 624,059. Arrays may also be synthesized by spotting monomers reagents on to a support using an ink jet printer. See id. and Pease, et al., EP 728,520.

cDNA probes may be prepared according to methods known in the art and further described herein, e.g., reverse-transcription PCR (RT-PCR) of RNA using sequence specific primers. Oligonucleotide probes may be synthesized chemically. Sequences of the genes or cDNA from which probes are made may be obtained, e.g., from GenBank, other public databases or publications.

Nucleic acid probes may be natural nucleic acids, chemically modified nucleic acids, e.g., composed of nucleotide analogs, as long as they have activated hydroxyl groups compatible with the linking chemistry. The protective groups can, themselves, be photolabile. Alternatively, the protective groups may be labile under certain chemical conditions, e.g., acid. In this example, the surface of the solid support may contain a composition that generates acids upon exposure to light. Thus, exposure of a region of the substrate to light generates acids in that region that remove the protective groups in the exposed region. Also, the synthesis method may use 3′-protected 5′-O-phosphoramidite-activated deoxynucleoside. In this case, the oligonucleotide is synthesized in the 5′ to 3′ direction, which results in a free 5′ end.

In one embodiment, oligonucleotides of an array are synthesized using a 96 well automated multiplex oligonucleotide synthesizer (A.M.O.S.) that is capable of making thousands of oligonucleotides (Lashkari, et al., PNAS, 1995, 93: 7912) may be used.

It will be appreciated that oligonucleotide design is influenced by the intended application. For example, it may be desirable to have similar melting temperatures for all of the probes. Accordingly, the length of the probes are adjusted so that the melting temperatures for all of the probes on the array are closely similar (it will be appreciated that different lengths for different probes may be needed to achieve a particular T[m] where different probes have different GC contents). Although melting temperature is a primary consideration in probe design, other factors are optionally used to further adjust probe construction, such as selecting against primer self-complementarity and the like.

Arrays, e.g., microarrrays, may conveniently be stored following fabrication or purchase for use at a later time. Under appropriate conditions, the subject arrays are capable of being stored for at least about 6 months and may be stored for up to one year or longer. Arrays are generally stored at temperatures between about −20° C. to room temperature, where the arrays are preferably sealed in a plastic container, e.g. bag, and shielded from light.

The next step is to contact the labeled nucleic acids with the array under conditions sufficient for binding between the probe and the target of the array. In a preferred embodiment, the probe will be contacted with the array under conditions sufficient for hybridization to occur between the labeled nucleic acids and probes on the microarray, where the hybridization conditions will be selected in order to provide for the desired level of hybridization specificity. Methods of using microarrays for detecting gene expression levels are described below in Section 6.

6. Methods for Detecting Gene Expression Levels

6.1. Use of Microarrays for Determining Gene Expression Levels

Generally, determining expression profiles with microarrays involves the following steps: (a) obtaining a mRNA sample from a subject and preparing labeled nucleic acids therefrom (the “target nucleic acids” or “targets”); (b) contact of the target nucleic acids with the array under conditions sufficient for target nucleic acids to bind with corresponding probe on the array, e.g. by hybridization or specific binding; (c) optional removal of unbound targets from the array; and (d) detection of bound targets, and analysis of the results, e.g., using computer based analysis methods. As used herein, “nucleic acid probes” or “probes” are nucleic acids attached to the array, whereas “target nucleic acids” are nucleic acids that are hybridized to the array. Each of these steps is described in more detail below.

(i) Obtaining a mRNA Sample of a Subject

Nucleic acid specimens may be obtained from an individual to be tested using either “invasive” or “non-invasive” sampling means. A sampling means is said to be “invasive” if it involves the collection of nucleic acids from within the skin or organs of an animal (including, especially, a murine, a human, an ovine, an equine, a bovine, a porcine, a canine, or a feline animal). Examples of invasive methods include needle biopsy, pleural aspiration, etc. Examples of such methods are discussed by Kim, C. H. et al., J. Virol., 1992, 66:3879-3882; Biswas, B. et al., Annals NY Acad. Sci., 1990, 590:582-583; Biswas, B., et al., J. Clin. Microbiol., 1991, 29:2228-2233. Extraction of adipose tissue from individuals used in some embodiments of this invention is well known to those skilled in the art, for example as described by Lonnroth, et al., Diabetes, 1983, 32980: 748-54.

In an embodiment the assays of the present invention will be performed on cells including but not limited to adipose cells from a mammal, adipocyte cultures propagated for laboratory purposes, 3T3-L1 adipocytes cells, cells of skeletal muscle derived from a mammal, skeletal muscle cells propagated for laboratory purposes, C2C12 myotube cells, etc. Primary cultures or cell lines can be used. Alternatively, embroyonic stem (ES) cells differentiated into adipocytes can be used, for example, as described in Poliard, et al., Journal of Cell Biology, 1995, 130: 1461-72. Appropriate cell lines that can be obtained for screening purposes are commercially available from the ATCC.

In one embodiment, one or more cells from the subject to be tested are obtained and RNA is isolated from the cells. In a preferred embodiment, a sample of adipose cells is obtained from the subject. When obtaining the cells, it is preferable to obtain a sample containing predominantly cells of the desired type, e.g., a sample of cells in which at least about 50%, preferably at least about 60%, even more preferably at least about 70%, 80% and even more preferably, at least about 90% of the cells are of the desired type. A higher percentage of cells of the desired type is preferable, since such a sample is more likely to provide clear gene expression data.

(ii) Hybridization of the Target Nucleic Acids to the Microarray

Contact of the array and probe involves contacting the array with an aqueous medium comprising the probe. Contact may be achieved in a variety of different ways depending on specific configuration of the array. For example, where the array simply comprises the pattern of size separated targets on the surface of a “plate-like” rigid substrate, contact may be accomplished by simply placing the array in a container comprising the probe solution, such as a polyethylene bag, and the like. In other embodiments where the array is entrapped in a separation media bounded by two rigid plates, the opportunity exists to deliver the probe via electrophoretic means. Alternatively, where the array is incorporated into a biochip device having fluid entry and exit ports, the probe solution may be introduced into the chamber in which the pattern of target molecules is presented through the entry port, where fluid introduction could be performed manually or with an automated device. In multiwell embodiments, the probe solution will be introduced in the reaction chamber comprising the array, either manually, e.g. with a pipette, or with an automated fluid handling device.

Contact of the probe solution and the targets will be maintained for a sufficient period of time for binding between the probe and the target to occur. Although dependent on the nature of the probe and target, contact will generally be maintained for a period of time ranging from about 10 min to 24 hrs, usually from about 30 min to 12 hrs and more usually from about 1 hr to 6 hrs.

When using commercially available microarrays, adequate hybridization conditions are provided by the manufacturer. When using non-commercial microarrays, adequate hybridization conditions may be determined based on the following hybridization guidelines, as well as on the hybridization conditions described in the numerous published articles on the use of microarrays.

Nucleic acid hybridization and wash conditions are optimally chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It may easily be demonstrated that specific hybridization conditions result in specific hybridization by carrying out a hybridization assay including negative controls.

Hybridization is carried out in conditions permitting essentially specific hybridization. The length of the probe and GC content will determine the Tm of the hybrid, and thus the hybridization conditions necessary for obtaining specific hybridization of the probe to the template nucleic acid. These factors are well known to a person of skill in the art, and may also be tested in assays. An extensive guide to the hybridization of nucleic acids is found in Tijssen (1993), “Laboratory Techniques in biochemistry and molecular biology-hybridization with nucleic acid probes.” Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Highly stringent conditions are selected to be equal to the Tm point for a particular probe. Sometimes the term “Td” is used to define the temperature at which at least half of the probe dissociates from a perfectly matched target nucleic acid. In any case, a variety of estimation techniques for estimating the Tm or Td are available, and generally described in Tijssen, supra. Typically, G-C base pairs in a duplex are estimated to contribute about 3° C. to the Tm, while A-T base pairs are estimated to contribute about 2° C., up to a theoretical maximum of about 80-100° C. However, more sophisticated models of Tm and Td are available and appropriate in which G-C stacking interactions, solvent effects, the desired assay temperature and the like are taken into account. For example, probes may be designed to have a dissociation temperature (Td) of approximately 60° C., using the formula: Td=(((((3×#GC)+(2×#AT))×37)−562)/#bp)−5; where #GC, #AT, and #bp are the number of guanine-cytosine base pairs, the number of adenine-thymine base pairs, and the number of total base pairs, respectively, involved in the annealing of the probe to the template DNA.

The stability difference between a perfectly matched duplex and a mismatched duplex, particularly if the mismatch is only a single base, may be quite small, corresponding to a difference in Tm between the two of as little as 0.5 degrees. See Tibanyenda, N., et al., Eur. J. Biochem., 1984, 139:19 and Ebel, S., et al., Biochem., 1992, 31:12083. More importantly, it is understood that as the length of the homology region increases, the effect of a single base mismatch on overall duplex stability decreases.

Theory and practice of nucleic acid hybridization is described, e.g., in S. Agrawal (ed.) Methods in Molecular Biology, volume 20; and Tijssen (1993) Laboratory Techniques in biochemistry and molecular biology-hybridization with nucleic acid probes, e.g., part I chapter 2 “Overview of principles of hybridization and the strategy of nucleic acid probe assays”, Elsevier, New York, provide a basic guide to nucleic acid hybridization.

Certain microarrays are of “active” nature, i.e., they provide independent electronic control over all aspects of the hybridization reaction (or any other affinity reaction) occurring at each specific microlocation. These devices provide a new mechanism for affecting hybridization reactions which is called electronic stringency control (ESC). The active devices of this invention may electronically produce “different stringency conditions” at each microlocation. Thus, all hybridizations may be carried out optimally in the same bulk solution. These arrays are described in U.S. Pat. No. 6,051,380 by Sosnowski et al.

In a preferred embodiment, background signal is reduced by the use of a detergent (e.g, C-TAB) or a blocking reagent (e.g., sperm DNA, cot-1 DNA, etc.) during the hybridization to reduce non-specific binding. In a particularly preferred embodiment, the hybridization is performed in the presence of about 0.5 mg/ml DNA (e.g., herring sperm DNA). The use of blocking agents in hybridization is well known to those of skill in the art (see, e.g., Chapter 8 in Laboratory Techniques in Biochemistry and Molecular Biology, Vol. 24: Hybridization With Nucleic Acid Probes, 1993, P. Tijssen, ed., Elsevier, N.Y.).

The method may or may not further comprise a non-bound label removal step prior to the detection step, depending on the particular label employed on the target nucleic acid. For example, in certain assay formats (e.g., “homogenous assay formats”) a detectable signal is only generated upon specific binding of target to probe. As such, in these assay formats, the hybridization pattern may be detected without a non-bound label removal step. In other embodiments, the label employed will generate a signal whether or not the target is specifically bound to its probe. In such embodiments, the non-bound labeled target is removed from the support surface. One means of removing the non-bound labeled target is to perform the well known technique of washing, where a variety of wash solutions and protocols for their use in removing non-bound label are known to those of skill in the art and may be used. Alternatively, non-bound labeled target may be removed by electrophoretic means.

Where all of the target sequences are detected using the same label, different arrays will be employed for each physiological source (where different could include using the same array at different times). The above methods may be varied to provide for multiplex analysis, by employing different and distinguishable labels for the different target populations (representing each of the different physiological sources being assayed). According to this multiplex method, the same array is used at the same time for each of the different target populations.

In another embodiment, hybridization is monitored in real time using a charge-coupled device imaging camera (Guschin, et al., Anal. Biochem., 1997, 250:203). Synthesis of arrays on optical fiber bundles allows easy and sensitive reading (Healy, et al., Anal. Biochem., 1997, 251:270). In another embodiment, real time hybridization detection is carried out on microarrays without washing using evanescent wave effect that excites only fluorophores that are bound to the surface (see, e.g., Stimpson, et al., PNAS, 1995 92:6379).

(iii) Detection of Hybridization and Analysis of Results

The above steps result in the production of hybridization patterns of labeled target nucleic acid on the array surface. The resultant hybridization patterns of labeled nucleic acids may be visualized or detected in a variety of ways, with the particular manner of detection being chosen based on the particular label of the target nucleic acid, where representative detection means include scintillation counting, autoradiography, fluorescence measurement, calorimetric measurement, light emission measurement, light scattering, and the like.

One method of detection includes an array scanner that is commercially available from Affymetrix (Santa Clara, Calif.), e.g., the 417™ Arrayer, the 418™ Array Scanner, or the Agilent GeneArray™ Scanner. This scanner is controlled from the system computer with a Windows^{R} interface and easy-to-use software tools. The output is a 16-bit.tif file that may be directly imported into or directly read by a variety of software applications. Preferred scanning devices are described in, e.g., U.S. Pat. Nos. 5,143,854 and 5,424,186.

When fluorescently labeled probes are used, the fluorescence emissions at each site of a transcript array may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores may be analyzed simultaneously (see Shalon et al., 1996, A DNA microarray system for analyzing complex DNA samples using two-color fluorescent probe hybridization, Genome Research, 6:639-645, which is incorporated by reference in its entirety for all purposes). In a preferred embodiment, the arrays are scanned with a laser fluorescent scanner with a computer controlled X-Y stage and a microscope objective. Sequential excitation of the two fluorophores may be achieved with a multi-line, mixed gas laser and the emitted light is split by wavelength and detected with two photomultiplier tubes. Fluorescence laser scanning devices are described in Schena, et al., 1996, Genome Res. 6:639-645 and in other references cited herein. Alternatively, the fiber-optic bundle described by Ferguson, et al., 1996, Nature Biotech. 14:1681-1684, may be used to monitor mRNA abundance levels.

In one embodiment in which fluorescent target nucleic acids are used, the arrays may be scanned using lasers to excite fluorescently labeled targets that have hybridized to regions of probe arrays, which may then be imaged using charged coupled devices (“CCDs”) for a wide field scanning of the array. Alternatively, another particularly useful method for gathering data from the arrays is through the use of laser confocal microscopy which combines the ease and speed of a readily automated process with high resolution detection.

Following the data gathering operation, the data will typically be reported to a data analysis operation. To facilitate the sample analysis operation, the data obtained by the reader from the device will typically be analyzed using a digital computer. Typically, the computer will be appropriately programmed for receipt and storage of the data from the device, as well as for analysis and reporting of the data gathered, e.g., subtraction of the background, deconvolution of multi-color images, flagging or removing artifacts, verifying that controls have performed properly, normalizing the signals, interpreting fluorescence data to determine the amount of hybridized target, normalization of background and single base mismatch hybridizations, and the like. In a preferred embodiment, a system comprises a search function that allows one to search for specific patterns, e.g., patterns relating to differential gene expression, e.g., between the expression profile of a cell of a subject having an erythropoietic disorder and the expression profile of a counterpart normal cell in a subject. A system preferably allows one to search for patterns of gene expression between more than two samples.

A desirable system for analyzing data is a general and flexible system for the visualization, manipulation, and analysis of gene expression data. Such a system preferably includes a graphical user interface for browsing and navigating through the expression data, allowing a user to selectively view and highlight the genes of interest. The system also preferably includes sort and search functions and is preferably available for general users with PC, Mac or Unix workstations. Also preferably included in the system are clustering algorithms that are qualitatively more efficient than existing ones. The accuracy of such algorithms is preferably hierarchically adjustable so that the level of detail of clustering may be systematically refined as desired.

Various algorithms are available for analyzing the gene expression profile data, e.g., the type of comparisons to perform. In certain embodiments, it is desirable to group genes that are co-regulated. This allows the comparison of large numbers of profiles. A preferred embodiment for identifying such groups of genes involves clustering algorithms (for reviews of clustering algorithms, see, e.g., Fukunaga, Statistical Pattern Recognition, 1990, 2nd Ed., Academic Press, San Diego; Everitt, Cluster Analysis, 1974, London: Heinemann Educ. Books; Hartigan, Clustering Algorithms, 1975, New York: Wiley; Sneath and Sokal, Numerical Taxonomy, 1973, Freeman; Anderberg, Cluster Analysis for Applications, 1973, Academic Press: New York).

Clustering analysis is useful in helping to reduce complex patterns of thousands of time curves into a smaller set of representative clusters. Some systems allow the clustering and viewing of genes based on sequences. Other systems allow clustering based on other characteristics of the genes, e.g., their level of expression (see, e.g., U.S. Pat. No. 6,203,987). Other systems permit clustering of time curves (see, e.g. U.S. Pat. No. 6,263,287). Cluster analysis may be performed using the hclust routine (see, e.g., “hclust” routine from the software package S-Plus, MathSoft, Inc., Cambridge, Mass.).

In some specific embodiments, genes are grouped according to the degree of co-variation of their transcription, presumably co-regulation, as described in U.S. Pat. No. 6,203,987. Groups of genes that have co-varying transcripts are termed “genesets.” Cluster analysis or other statistical classification methods may be used to analyze the co-variation of transcription of genes in response to a variety of perturbations, e.g. caused by a disease or a drug. In one specific embodiment, clustering algorithms are applied to expression profiles to construct a “similarity tree” or “clustering tree” which relates genes by the amount of co-regulation exhibited. Genesets are defined on the branches of a clustering tree by cutting across the clustering tree at different levels in the branching hierarchy.

In some embodiments, a gene expression profile is converted to a projected gene expression profile. The projected gene expression profile is a collection of geneset expression values. The conversion is achieved, in some embodiments, by averaging the level of expression of the genes within each geneset. In some other embodiments, other linear projection processes may be used. The projection operation expresses the profile on a smaller and biologically more meaningful set of coordinates, reducing the effects of measurement errors by averaging them over each cellular constituent sets and aiding biological interpretation of the profile.

In one embodiment, RNA is obtained from a single cell. It is also possible to obtain cells from a subject and culture the cells in vitro, such as to obtain a larger population of cells from which RNA may be extracted. Methods for establishing cultures of non-transformed cells, i.e., primary cell cultures, are known in the art. It is also possible to obtain a cell sample from a subject, and then to enrich it in the desired cell type. For example, cells may be isolated from other cells using a variety of techniques, such as isolation with an antibody binding to an epitope on the cell surface of the desired cell type.

When isolating RNA from tissue samples or cells from individuals, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells may quickly become degraded. Accordingly, in a preferred embodiment, the cells obtained from a subject are snap frozen as soon as possible.

RNA may be extracted from the tissue sample by a variety of methods, e.g., the guanidium thiocyanate lysis followed by CsCl centrifugation (Chirgwin, et al., Biochemistry, 1979,18:5294-5299). RNA from single cells may be obtained as described in methods for preparing cDNA libraries from single cells, such as those described in Dulac, C., Curr. Top. Dev. Biol., 1998, 36, 245 and Jena, et al., J. Immunol. Methods, 1996, 190:199. Care to avoid RNA degradation must be taken, e.g., by inclusion of RNAsin.

The RNA sample may then be enriched in particular species. In one embodiment, poly(A)+ RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.).

In a preferred embodiment, the RNA population is enriched in sequences of interest. Enrichment may be undertaken, e.g., by primer-specific cDNA synthesis, or multiple rounds of linear amplification based on cDNA synthesis and template-directed in vitro transcription (see, e.g., Wang, et al., PNAS, 1998, 86, 9717; Dulac, et al., supra, and Jena, et al., supra).

The population of RNA, enriched or not in particular species or sequences, may further be amplified. Such amplification is particularly important when using RNA from a single or a few cells. A variety of amplification methods are suitable for use in the methods of the invention, including, e.g., PCR; ligase chain reaction (LCR) (see, e.g., Wu and Wallace, Genomics, 1998, 4, 560, Landegren, et al., Science, 1998, 241, 1077; self-sustained sequence replication (SSR) (see, e.g., Guatelli, et al., Proc. Nat. Acad. Sci. USA, 1990, 87, 1874; nucleic acid based sequence amplification (NASBA) and transcription amplification (see, e.g., Kwoh, et al., Proc. Natl. Acad. Sci. USA, 1989, 86,1173. For PCR technology, see, e.g., PCR Technology: Principles and Applications for DNA Amplification, 1992, ed. H. A. Erlich, Freeman Press, N.Y., N.Y.; PCR Protocols: A Guide to Methods and applications, eds. Innis, et al., Academic Press, San Diego, Calif., 1990; Mattila, et al., Nucleic Acids Res., 1991,19, 4967; Eckert, et al., PCR Methods and Applications 1, 17 (1991); PCR (eds. McPherson, et al., IRL Press, Oxford); and U.S. Pat. No. 4,683,202. Methods of amplification are described, e.g., in Ohyama, et al., BioTechniques, 2000, 29:530; Luo, et al., Nat. Med. 5, 1999,117; Hegde, et al., BioTechniques, 2000, 29:548; Kacharmina, et al., Meth. Enzymol., 1999, 303:3; Livesey, et al., Curr. Biol., 2000,10:301; Spirin, et al. Invest. Ophtalmol. Vis. Sci., 1999, 40:3108; and Sakai, et al., Anal. Biochem., 2000, 287:32. RNA amplification and cDNA synthesis may also be conducted in cells in situ (see, e.g., Eberwine, et al. PNAS, 1992, 89:3010).

One of skill in the art will appreciate that whatever amplification method is used, if a quantitative result is desired, care must be taken to use a method that maintains or controls for the relative frequencies of the amplified nucleic acids to achieve quantitative amplification. Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. A high density array may then include probes specific to the internal standard for quantification of the amplified nucleic acid.

One preferred internal standard is a synthetic AW106 cRNA. The AW106 cRNA is combined with RNA isolated from the sample according to standard techniques known to those of skilled in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of radioactivity (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al., Academic Press, Inc. N.Y., 1990.

In a preferred embodiment, a sample mRNA is reverse transcribed with a reverse transcriptase and a primer consisting of oligo(dT) and a sequence encoding the phage T7 promoter to provide single stranded DNA template. The second DNA strand is polymerized using a DNA polymerase. After synthesis of double-stranded cDNA, T7 RNA polymerase is added and RNA is transcribed from the cDNA template. Successive rounds of transcription from each single cDNA template results in amplified RNA. Methods of in vitro polymerization are well known to those of skill in the art (see, e.g., Sambrook, (supra) and this particular method is described in detail by Van Gelder, et al., Proc. Natl. Acad. Sci. USA, 1990, 87: 1663-1667, who demonstrate that in vitro amplification according to this method preserves the relative frequencies of the various RNA transcripts. Moreover, Eberwine, et al. Proc. Natl. Acad. Sci. USA, 89: 3010-3014 provide a protocol that uses two rounds of amplification via in vitro transcription to achieve greater than 10⁶ fold amplification of the original starting material, thereby permitting expression monitoring even where biological samples are limited.

It will be appreciated by one of skill in the art that the direct transcription method described above provides an antisense (aRNA) pool. Where antisense RNA is used as the target nucleic acid, the oligonucleotide probes provided in the array are chosen to be complementary to subsequences of the antisense nucleic acids. Conversely, where the target nucleic acid pool is a pool of sense nucleic acids, the oligonucleotide probes are selected to be complementary to subsequences of the sense nucleic acids. Finally, where the nucleic acid pool is double stranded, the probes may be of either sense as the target nucleic acids include both sense and antisense strands.

Generally, the target molecules will be labeled to permit detection of hybridization of target molecules to a microarray. By labeled is meant that the probe comprises a member of a signal producing system and is thus detectable, either directly or through combined action with one or more additional members of a signal producing system. Examples of directly detectable labels include isotopic and fluorescent moieties incorporated into, usually covalently bonded to, a moiety of the probe, such as a nucleotide monomeric unit, e.g. dNMP of the primer, or a photoactive or chemically active derivative of a detectable label which may be bound to a functional moiety of the probe molecule.

Nucleic acids may be labeled after or during enrichment and/or amplification of RNAs. For example, labeled cDNA is prepared from mRNA by oligo dt-primed or random-primed reverse transcription, both of which are well known in the art (see, e.g., Klug and Berger, Methods Enzymol., 1987, 152:316-325). Reverse transcription may be carried out in the presence of a dNTP conjugated to a detectable label, most preferably a fluorescently labeled dNTP. Alternatively, isolated mRNA may be converted to labeled antisense RNA synthesized by in vitro transcription of double-stranded cDNA in the presence of labeled dNTPs (Lockhart et al., 1996, “Expression monitoring by hybridization to high-density oligonucleotide arrays,” Nature Biotech., 14:1675, which is incorporated by reference in its entirety for all purposes).

In alternative embodiments, the cDNA or RNA probe may be synthesized in the absence of detectable label and may be labeled subsequently, e.g., by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent.

In one embodiment, labeled cDNA is synthesized by incubating a mixture containing 0.5 mM dGTP, dATP and dCTP plus 0.1 mM dTTP plus fluorescent deoxyribonucleotides (e.g., 0.1 mM Rhodamine 110 UTP (Perken Elmer Cetus) or 0.1 mM Cy3 dUTP (Amersham)) with reverse transcriptase (e.g., SuperScript.™.II, LTI Inc.) at 42° C. for 60 minutes.

Fluorescent moieties or labels of interest include coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. Texas red, tetramethylrhodamine, eosins and erythrosins, cyanine dyes, e.g. Cy2, Cy3, Cy3.5, Cy5, Cy5.5, Cy7, Fluor X, macrocyclic chelates of lanthanide ions, e.g. quantum dye™, fluorescent energy transfer dyes, such as thiazole orange-ethidium heterodimer, TOTAB, dansyl, etc. Individual fluorescent compounds which have functionalities for linking to an element desirably detected in an apparatus or assay of the invention, or which may be modified to incorporate such functionalities include, e.g., dansyl chloride; fluoresceins such as 3,6-dihydroxy-9-phenylxanthydrol; rhodamineisothiocyanate; N-phenyl 1-amino-8-sulfonatonaphthalene; N-phenyl 2-amino-6-sulfonatonaphthalene; 4-acetamido-4-isothiocyanato-stilbene-2,2′-disulfonic acid; pyrene-3-sulfonic acid; 2-toluidinonaphthalene-6-sulfonate; N-phenyl-N-methyl-2-aminoaphthalene-6-sulfonate; ethidium bromide; stebrine; auromine-0,2-(9′-anthroyl)palmitate; dansyl phosphatidylethanolamine; N,N′-dioctadecyl oxacarbocyanine: N,N′-dihexyl oxacarbocyanine; merocyanine, 4-(3′-pyrenyl)stearate; d-3-aminodesoxy-equilenin; 12-(9′-anthroyl)stearate; 2-methylanthracene; 9-vinylanthracene; 2,2′(vinylene-p-phenylene)bisbenzoxazole; p-bis(2-∃-methyl-5-phenyl-oxazolyl))benzene; 6-dimethylamino-1,2-benzophenazin; retinol; bis(3′-aminopyridinium) 1,10-decandiyl diiodide; sulfonaphthylhydrazone of hellibrienin; chlorotetracycline; N-(7-dimethylamino-4-methyl-2-oxo-3-chromenyl)maleimide; N-(p-(2benzimidazolyl)-phenyl)maleimide; N-(4-fluoranthyl)maleimide; bis(homovanillic acid); resazarin; 4-chloro-7-nitro-2,1,3-benzooxadiazole; merocyanine 540; resorufin; rose bengal; and 2,4-diphenyl-3(2H)-furanone. (see, e.g., Kricka, 1992, Nonisotopic DNA Probe Techniques, Academic Press San Diego, Calif.). Many fluorescent tags are commercially available from SIGMA chemical company (Saint Louis, Mo.), Amersham, Molecular Probes, R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), and Applied Biosystems (Foster City, Calif.) as well as other commercial sources known to one of skill.

Chemiluminescent labels include luciferin and 2,3-dihydrophthalazinediones, e.g., luminol.

Isotopic moieties or labels of interest include ³²P, ³³P, ³⁵S, ¹²⁵I, ²H, ¹⁴C, and the like (see Zhao, et al., “High density cDNA filter analysis: a novel approach for large-scale, quantitative analysis of gene expression,” Gene, 1995, 156:207; Pietu, et al., “Novel gene transcripts preferentially expressed in human muscles revealed by quantitative hybridization of a high density cDNA array,” Genome Res., 1996,6:492). However, because of scattering of radioactive particles, and the consequent requirement for widely spaced binding sites, use of radioisotopes is a less-preferred embodiment.

Labels may also be members of a signal producing system that act in concert with one or more additional members of the same system to provide a detectable signal. Illustrative of such labels are members of a specific binding pair, such as ligands, e.g. biotin, fluorescein, digoxigenin, antigen, polyvalent cations, chelator groups and the like, where the members specifically bind to additional members of the signal producing system, where the additional members provide a detectable signal either directly or indirectly, e.g. antibody conjugated to a fluorescent moiety or an enzymatic moiety capable of converting a substrate to a chromogenic product, e.g. alkaline phosphatase conjugate antibody and the like.

Additional labels of interest include those that provide for signal only when the probe with which they are associated is specifically bound to a target molecule, where such labels include: “molecular beacons” as described in Tyagi & Kramer, Nature Biotechnology, 1996, 14:303 and EP 0 070 685 B1. Other labels of interest include those described in U.S. Pat. No. 5,563,037; WO 97/17471 and WO 97/17076.

In some cases, hybridized target nucleic acids may be labeled following hybridization. For example, where biotin labeled dNTPs are used in, e.g., amplification or transcription, streptavidin linked reporter groups may be used to label hybridized complexes.

In other embodiments, the target nucleic acid is not labeled. In this case, hybridization may be determined, e.g., by plasmon resonance, as described, e.g., in Thiel, et al., Anal. Chem., 1997, 69:4948.

In one embodiment, a plurality (e.g., 2, 3, 4, 5 or more) of sets of target nucleic acids are labeled and used in one hybridization reaction (“multiplex” analysis). For example, one set of nucleic acids may correspond to RNA from one cell and another set of nucleic acids may correspond to RNA from another cell. The plurality of sets of nucleic acids may be labeled with different labels, e.g., different fluorescent labels which have distinct emission spectra so that they may be distinguished. The sets may then be mixed and hybridized simultaneously to one microarray.

For example, the two different cells may be an adipose cell treated with a PPARγ ligand and a counterpart adipose cell not treated with the PPARγ ligand. The cDNA derived from each of the two cell types are differently labeled so that they may be distinguished. In one embodiment, for example, cDNA from the adipose cell treated with PPARγ ligand is synthesized using a fluorescein-labeled dNTP, and cDNA from the second cell, i.e., the control adipose cell not treated with the PPARγ ligand, is synthesized using a rhodamine-labeled dNTP. When the two cDNAs are mixed and hybridized to the microarray, the relative intensity of signal from each cDNA set is determined for each site on the array, and any relative difference in abundance of a particular mRNA detected.

In the example described above, the cDNA from the adipose cell treated with a PPARγ ligand will fluoresce green when the fluorophore is stimulated and the cDNA from the adipose cell not treated with a PPARγ ligand will fluoresce red. As a result, if the two cells are essentially the same, the particular mRNA will be equally prevalent in both cells and, upon reverse transcription, red-labeled and green-labeled cDNA will be equally prevalent. When hybridized to the microarray, the binding site(s) for that species of RNA will emit wavelengths characteristic of both fluorophores (and appear brown in combination). In contrast, if the two cells are different, the ratio of green to red fluorescence will be different.

The use of a two-color fluorescence labeling and detection scheme to define alterations in gene expression has been described, e.g., in Shena, et al., “Quantitative monitoring of gene expression patterns with a complementary DNA microarray,” Science, 1995, 270:467-470. An advantage of using cDNA labeled with two different fluorophores is that a direct and internally controlled comparison of the mRNA levels corresponding to each arrayed gene in two cell states may be made, and variations due to minor differences in experimental conditions (e.g, hybridization conditions) will not affect subsequent analyses.

Examples of distinguishable labels for use when hybridizing a plurality of target nucleic acids to one array are well known in the art and include: two or more different emission wavelength fluorescent dyes, like Cy3 and Cy5, combination of fluorescent proteins and dyes, like phicoerythrin and Cy5, two or more isotopes with different energy of emission, like ³²P and ³³P, gold or silver particles with different scattering spectra, labels which generate signals under different treatment conditions, like temperature, pH, treatment by additional chemical agents, etc., or generate signals at different time points after treatment. Using one or more enzymes for signal generation allows for the use of an even greater variety of distinguishable labels, based on different substrate specificity of enzymes (alkaline phosphatase/peroxidase).

Further, it is preferable in order to reduce experimental error to reverse the fluorescent labels in two-color differential hybridization experiments to reduce biases peculiar to individual genes or array spot locations. In other words, it is preferable to first measure gene expression with one labeling (e.g., labeling nucleic acid from a first cell with a first fluorochrome and nucleic acid from a second cell with a second fluorochrome) of the mRNA from the two cells being measured, and then to measure gene expression from the two cells with reversed labeling (e.g., labeling nucleic acid from the first cell with the second fluorochrome and nucleic acid from the second cell with the first fluorochrome). Multiple measurements over exposure levels and perturbation control parameter levels provide additional experimental error control.

The quality of labeled nucleic acids may be evaluated prior to hybridization to an array. For example, a sample of the labeled nucleic acids may be hybridized to probes derived from the 5′, middle and 3′ portions of genes known to be or suspected to be present in the nucleic acid sample. This will be indicative as to whether the labeled nucleic acids are full length nucleic acids or whether they are degraded. In one embodiment, the GeneChipe Test3 Array from Affymetrix (Santa Clara, Calif.) may be used for that purpose. This array contains probes representing a subset of characterized genes from several organisms including mammals. Thus, the quality of a labeled nucleic acid sample may be determined by hybridization of a fraction of the sample to an array, such as the GeneChipe Test3 Array from Affymetrix (Santa Clara, Calif.).

6.2. Other Methods for Determining Gene Expression Levels

In certain embodiments, it is sufficient to determine the expression of one or only a few genes, as opposed to hundreds or thousands of genes. Although microarrays may be used in these embodiments, various other methods of detection of gene expression are available. This section describes a few exemplary methods for detecting and quantifying mRNA or polypeptide encoded thereby. Where the first step of the methods includes isolation of mRNA from cells, this step may be conducted as described above. Labeling of one or more nucleic acids may be performed as described above.

In one embodiment, mRNA obtained from a sample is reverse transcribed into a first cDNA strand and subjected to PCR, e.g., RT-PCR. House keeping genes, or other genes whose expression does not vary may be used as internal controls and controls across experiments. Following the PCR reaction, the amplified products may be separated by electrophoresis and detected. By using quantitative PCR, the level of amplified product will correlate with the level of RNA that was present in the sample. The amplified samples may also be separated on a agarose or polyacrylamide gel, transferred onto a filter, and the filter hybridized with a probe specific for the gene of interest. Numerous samples may be analyzed simultaneously by conducting parallel PCR amplification, e.g., by multiplex PCR.

In another embodiment, mRNA levels is determined by dotblot analysis and related methods (see, e.g., G. A. Beltz, et al., in Methods in Enzmmology, Vol. 100, Part B, R. Wu, L. Grossmam, K. Moldave, Eds., Academic Press, New York, Chapter 19, pp. 266-308, 1985). In one embodiment, a specified amount of RNA extracted from cells is blotted (i.e., non-covalently bound) onto a filter, and the filter is hybridized with a probe of the gene of interest. Numerous RNA samples may be analyzed simultaneously, since a blot may comprise multiple spots of RNA. Hybridization is detected using a method that depends on the type of label of the probe. In another dotblot method, one or more probes of one or more genes from Tables I and II are attached to a membrane, and the membrane is incubated with labeled nucleic acids obtained from and optionally derived from RNA of a cell or tissue of a subject. Such a dotblot is essentially an array comprising fewer probes than a microarray.

“Dot blot” hybridization gained wide-spread use, and many versions were developed (see, e.g., M. L. M. Anderson and B. D. Young, in Nucleic Acid Hybridization—A Practical Approach, B. D. Hames and S. J. Higgins, Eds., IRL Press, Washington D.C., Chapter 4, pp. 73-111,1985).

Another format, the so-called “sandwich” hybridization, involves covalently attaching oligonucleotide probes to a solid support and using them to capture and detect multiple nucleic acid targets (see, e.g., M. Ranki, et al., Gene, 21, pp. 77-85,1983; A. M. Palva, T. M. Ranki, and H. E. Soderlund, in UK Patent Application GB 2156074A, Oct. 2, 1985; T. M. Ranki and H. E. Soderlund in U.S. Pat. No. 4,563,419, Jan. 7, 1986; A. D. B. Malcolm and J. A. Langdale, in PCT WO 86/03782, Jul. 3, 1986; Y. Stabinsky, in U.S. Pat. No. 4,751,177, Jan. 14, 1988; T. H. Adams et al., in PCT WO 90/01564, Feb. 22, 1990; R. B. Wallace et al. 6 Nucleic Acid Res. 11, p. 3543,1979; and B. J. Connor, et al., 80 Proc. Natl. Acad. Sci. USA pp. 278-282, 1983). Multiplex versions of these formats are called “reverse dot blots.”

mRNA levels may also be determined by Northern blots. Specific amounts of RNA are separated by gel electrophoresis and transferred onto a filter which is then hybridized with a probe corresponding to the gene of interest. This method, although more burdensome when numerous samples and genes are to be analyzed provides the advantage of being very accurate.

A preferred method for high throughput analysis of gene expression is the serial analysis of gene expression (SAGE) technique, first described in Velculescu, et al., Science, 1995, 270, 484-487. Among the advantages of SAGE is that it has the potential to provide detection of all genes expressed in a given cell type, provides quantitative information about the relative expression of such genes, permits ready comparison of gene expression of genes in two cells, and yields sequence information that may be used to identify the detected genes. Thus far, SAGE methodology has proved itself to reliably detect expression of regulated and nonregulated genes in a variety of cell types (Velculescu, et al., 1997, Cell, 88, 243-251; Zhang, et al., Science, 1997, 276,1268-1272 and Velculescu, et al., Nat Genet, 1999, 23, 387-388.

Techniques for producing and probing nucleic acids are further described, for example, in Sambrook, et al., Molecular Cloning: A Laboratory Manual (New York, Cold Spring Harbor Laboratory, 1989).

Alternatively, the level of expression of one or more genes from Tables I and II is determined by in situ hybridization. In one embodiment, a tissue sample is obtained from a subject, the tissue sample is sliced, and in situ hybridization is performed according to methods known in the art, to determine the level of expression of the genes of interest.

Alternatively, the assaying of the modulation of gene expression via can be performed using a Real Time-PCR assay. Total mRNA is extracted as described above and subjected to the reverse transcription using an RNA-directed DNA polymerase, such as reverse transcriptase isolated from AMV, MoMuLV or recombinantly produced. The cDNAs produced by the lafter procedure can be amplified in the presence of Taq polymerase and the amplification monitored in an appropriate apparatus in real time as a function of PCR cycle number under the appropriate conditions that yield measurable signals, for example, in the presence of dyes that yield a particular absorbance reading when bound to duplex DNA. The relative concentrations of the mRNAs corresponding to chosen genes can be calculated from the cycle midpoints of their respective Real Time-PCR amplification curves and compared between cells exposed to a candidate therapeutic relative to a control cell in order to determine the increase or decrease in mRNA levels in a quantitative fashion.

In other methods, the level of expression of a gene is detected by measuring the level of protein encoded by the gene. This may be done, e.g., by immunoprecipitation, ELISA, or immunohistochemistry using an agent, e.g., an antibody, that specifically detects the protein encoded by the gene. Other techniques include Western blot analysis. Immunoassays are commonly used to quantitate the levels of proteins in cell samples, and many other immunoassay techniques are known in the art. The invention is not limited to a particular assay procedure, and therefore is intended to include both homogeneous and heterogeneous procedures. Exemplary immunoassays which may be conducted according to the invention include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, may be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art.

Alternatively, the assaying of the modulation of gene expression can be conducted by assaying for the protein levels in the cells. The cells can be lysed and serial dilutions of the extracts can be subjected to SDS gel electrophoresis. Levels of target protein between different cell cultures exposed to a candidate therapeutic relative to control cells can be compared between serial dilutions of extracts using the Western blot assay in a semi-quantitative manner.

In the case of polypeptides which are secreted from cells, the level of expression of these polypeptides may be measured in biological fluids.

6.3. Data Analysis Methods

Comparison of the expression levels of one or more genes from Tables I-IV in adipose cells in response to treatment with a PPARγ ligand with reference expression levels, e.g., expression levels in adipose cells not treated with the PPARγ ligand, is preferably conducted using computer systems. In one embodiment, expression levels are obtained in two cells and these two sets of expression levels are introduced into a computer system for comparison. In a preferred embodiment, one set of expression levels is entered into a computer system for comparison with values that are already present in the computer system, or in computer-readable form that is then entered into the computer system.

In one embodiment, the invention provides a computer readable form of the gene expression profile data of the invention, or of values corresponding to the level of expression of at least one gene from Tables I-IV from the adipose cell treated with the PPARγ ligand. The values may be mRNA expression levels obtained from experiments, e.g., microarray analysis. The values may also be mRNA levels normalized relative to a reference gene whose expression is constant in numerous cells under numerous conditions, e.g., GAPDH. In other embodiments, the values in the computer are ratios of, or differences between, normalized or non-normalized mRNA levels in different samples.

The gene expression profile data may be in the form of a table, such as an Excel table. The data may be alone, or it may be part of a larger database, e.g., comprising other expression profiles. For example, the expression profile data of the invention may be part of a public database. The computer readable form may be in a computer. In another embodiment, the invention provides a computer displaying the gene expression profile data.

In one embodiment, the invention provides a method for determining the similarity between the level of expression of one or more genes from Tables I-IV in a first cell, e.g., an adipose cell of a subject treated with a PPARγ ligand, and that in a second cell, comprising obtaining the level of expression of one or more genes from Tables I-IV in a first cell and entering these values into a computer comprising a database including records comprising values corresponding to levels of expression of one or more genes from Tables I-IV in a second cell, and processor instructions, e.g., a user interface, capable of receiving a selection of one or more values for comparison purposes with data that is stored in the computer. The computer may further comprise a means for converting the comparison data into a diagram or chart or other type of output.

In another embodiment, values representing expression levels of genes from Tables I-IV are entered into a computer system, comprising one or more databases with reference expression levels obtained from more than one cell. For example, the computer comprises expression data of adipose cells that are treated or not treated with a PPARγ ligand. Instructions are provided to the computer, and the computer is capable of comparing the data entered with the data in the computer to determine whether the data entered is more similar to that of an adipose cell that is treated or not treated with a PPARγ ligand.

In another embodiment, the computer comprises values of expression levels in cells of subjects at different stages of treatment with a PPARγ ligand and the computer is capable of comparing expression data entered into the computer with the data stored, and produce results indicating to which of the expression profiles in the computer, the one entered is most similar, such as to determine the decline of responsiveness to treatment or the development of side effects of the treatment in the subject.

In yet another embodiment, the reference expression profiles in the computer are expression profiles from cells of one or more subjects undergoing treatment with a PPARγ ligand, which cells are treated in vivo or in vitro with a PPARγ ligand used for therapy of a disease associated with PPARγ, such as Type II diabetes. Upon entering of expression data of a cell of a subject treated in vitro or in vivo with the PPARγ ligand, the computer is instructed to compare the data entered to the data in the computer, and to provide results indicating whether the expression data input into the computer are more similar to those of a cell of a subject that is responsive to the drug or more similar to those of a cell of a subject that is not responsive to the drug. Thus, the results indicate whether the subject is likely to respond to the treatment with the drug or unlikely to respond to it.

In one embodiment, the invention provides a system that comprises a means for receiving gene expression data for one or a plurality of genes; a means for comparing the gene expression data from each of said one or plurality of genes to a common reference frame; and a means for presenting the results of the comparison. This system may further comprise a means for clustering the data.

In another embodiment, the invention provides a computer program for analyzing gene expression data comprising (i) a computer code that receives as input gene expression data for a plurality of genes and (ii) a computer code that compares said gene expression data from each of said plurality of genes to a common reference frame.

The invention also provides a machine-readable or computer-readable medium including program instructions for performing the following steps: (i) comparing a plurality of values corresponding to expression levels of one or more genes from Tables I-IV in a query cell with a database including records comprising reference expression or expression profile data of one or more reference cells and an annotation of the type of cell; and (ii) indicating to which cell the query cell is most similar based on similarities of expression profiles. The reference cells may be cells from subjects at different stages in the treatment with a PPARγ ligand.

The reference cells may also be cells from subjects responding or not responding to several different treatments with PPARγ ligands, and the computer system indicates a preferred treatment for the subject. Accordingly, the invention provides a method for selecting a therapy for a patient having a disease associated with the PPARγ receptor; the method comprising: (i) providing the levels of expression of one or more genes from Tables I-IV from adipose cells of the patient cultured with various PPARγ ligands; (ii) providing a plurality of reference profiles, each associated with a therapy, wherein the subject expression profiles and each reference profile has a plurality of values, each value representing the level of expression of a gene from Tables I-IV; and (iii) selecting the reference profile most similar to the subject expression profile, to thereby select a therapy for said patient. In a preferred embodiment step (iii) is performed by a computer. The most similar reference profile may be selected by weighing a comparison value of the plurality using a weight value associated with the corresponding expression data.

The relative abundance of a mRNA in two biological samples may be scored as a perturbation and its magnitude determined (i.e., the abundance is different in the two sources of mRNA tested), or as not perturbed (i.e., the relative abundance is the same). In various embodiments, a difference between the two sources of RNA of at least a factor of about 25% (RNA from one source is 25% more abundant in one source than the other source), more usually about 50%, even more often by a factor of about 2 (twice as abundant), 3 (three times as abundant) or 5 (five times as abundant) is scored as a perturbation. Perturbations may be used by a computer for calculating and expression comparisons.

Preferably, in addition to identifying a perturbation as positive or negative, it is advantageous to determine the magnitude of the perturbation. This may be carried out, as noted above, by calculating the ratio of the emission of the two fluorophores used for differential labeling, or by analogous methods that will be readily apparent to those of skill in the art.

In operation, the means for receiving gene expression data, the means for comparing the gene expression data, the means for presenting, the means for normalizing, and the means for clustering within the context of the systems of the present invention may involve a programmed computer with the respective functionalities described herein, implemented in hardware or hardware and software; a logic circuit or other component of a programmed computer that performs the operations specifically identified herein, dictated by a computer program; or a computer memory encoded with executable instructions representing a computer program that may cause a computer to function in the particular fashion described herein.

Those skilled in the art will understand that the systems and methods of the present invention may be applied to a variety of systems, including IBM-compatible personal computers running MS-DOS or Microsoft Windows.

The computer may have internal components linked to external components. The internal components may include a processor element interconnected with a main memory. The computer system may be an Intel Pentium®-based processor of 200 MHz or greater clock rate and with 32 MB or more of main memory. The external component may comprise a mass storage, which may be one or more hard disks (which are typically packaged together with the processor and memory). Such hard disks are typically of 1 GB or greater storage capacity. Other external components include a user interface device, which may be a monitor, together with an inputing device, which may be a “mouse”, or other graphic input devices, and/or a keyboard. A printing device may also be attached to the computer.

Typically, the computer system is also linked to a network link, which may be part of an Ethernet link to other local computer systems, remote computer systems, or wide area communication networks, such as the Internet. This network link allows the computer system to share data and processing tasks with other computer systems.

Loaded into memory during operation of this system are several software components, which are both standard in the art and special to the instant invention. These software components collectively cause the computer system to function according to the methods of this invention. These software components are typically stored on a mass storage. A software component represents the operating system, which is responsible for managing the computer system and its network interconnections. This operating system may be, for example, of the Microsoft Windows' family, such as Windows 95, Windows 98, or Windows NT. A software component represents common languages and functions conveniently present on this system to assist programs implementing the methods specific to this invention. Many high or low level computer languages may be used to program the analytic methods of this invention. Instructions may be interpreted during run-time or compiled. Preferred languages include C/C++, and JAVA®. Most preferably, the methods of this invention are programmed in mathematical software packages which allow symbolic entry of equations and high-level specification of processing, including algorithms to be used, thereby freeing a user of the need to procedurally program individual equations or algorithms. Such packages include Matlab from Mathworks (Natick, Mass.), Mathematica from Wolfram Research (Champaign, Ill.), or S-Plus from Math Soft (Cambridge, Mass.). Accordingly, a software component represents the analytic methods of this invention as programmed in a procedural language or symbolic package. In a preferred embodiment, the computer system also contains a database comprising values representing levels of expression of one or more genes from Tables I-IV. The database may contain one or more expression profiles of genes whose expression is characteristic of treatment with a PPARγ ligand.

In an exemplary implementation, to practice the methods of the present invention, a user first loads expression profile data into the computer system. These data may be directly entered by the user from a monitor and keyboard, or from other computer systems linked by a network connection, or on removable storage media such as a CD-ROM or floppy disk or through the network. Next the user causes execution of expression profile analysis software which performs the steps of comparing and, e.g., clustering co-varying genes into groups of genes.

In another exemplary implementation, expression profiles are compared using a method described in U.S. Pat. No. 6,203,987. A user first loads expression profile data into the computer system. Geneset profile definitions are loaded into the memory from the storage media or from a remote computer, preferably from a dynamic geneset database system, through the network. Next the user causes execution of projection software which performs the steps of converting expression profile to projected expression profiles. The projected expression profiles are then displayed.

In yet another exemplary implementation, a user first leads a projected profile into the memory. The user then causes the loading of a reference profile into the memory. Next, the user causes the execution of comparison software which performs the steps of objectively comparing the profiles.

6.4. Diagnostic and Prognostic Compositions and Devices

Any composition and device (e.g., a microarray) used in the above-described methods are within the scope of the invention.

In one embodiment, the invention provides a composition comprising a plurality of detection agents for detecting expression of genes in Tables I-IV. In a preferred embodiment, the composition comprises at least 1, preferably at least 3, 5, 10, 20, 50 or all 54 different detection agents. A detection agent may be a nucleic acid probe, e.g., DNA or RNA, or it may be a polypeptide, e.g., as antibody that binds to the polypeptide encoded by a gene listed in Tables I-V. The probes may be present in equal amount or in different amounts in the solution.

A nucleic acid probe may be at least about 10 nucleotides long, preferably at least about 15, 20, 25, 30, 50, 100 nucleotides or more, and may comprise the full length gene. Preferred probes are those that hybridize specifically to genes listed in Tables I-V. If the nucleic acid is short (i.e., 20 nucleotides or less), the sequence is preferably perfectly complementary to the target gene (i.e., a gene from Tables I-IV), such that specific hybridization may be obtained. However, nucleic acids, even short ones, which are not perfectly complementary to the target gene, may also be included in a composition of the invention, e.g., for use as a negative control. Certain compositions may also comprise nucleic acids that are complementary to, and capable of detecting, an allele of a gene.

In a preferred embodiment, the invention provides nucleic acids which hybridize under high stringency conditions of 0.2 to 1×SSC at 65° C. followed by a wash at 0.2×SSC at 65° C. to genes from Tables I-IV. In another embodiment, the invention provides nucleic acids which hybridize under low stringency conditions of 6×SSC at room temperature followed by a wash at 2×SSC at room temperature. Other nucleic acids probes hybridize to their target in 3×SSC at 40 or 50° C., followed by a wash in 1 or 2×SSC at 20, 30, 40, 50, 60, or 65° C.

Nucleic acids which are at least about 80%, preferably at least about 90%, even more preferably at least about 95% and most preferably at least about 98% identical to genes from Tables I and II or cDNAs thereof, and complements thereof, are also within the scope of the invention.

Nucleic acid probes may be obtained by, e.g., polymerase chain reaction (PCR) amplification of gene segments from genomic DNA, cDNA (e.g., by RT-PCR), or cloned sequences. PCR primers are chosen, based on the known sequence of the genes or cDNA, that result in amplification of unique fragments. Computer programs may be used in the design of primers with the required specificity and optimal amplification properties. See, e.g., Oligo version 5.0 (National Biosciences). Factors which apply to the design and selection of primers for amplification are described, for example, by Rylchik, W., “Selection of Primers for Polymerase Chain Reaction,” in Methods in Molecular Biology, 1993, vol. 15, White B. ed., Humana Press, Totowa, N.J. Sequences may be obtained from GenBank or other public sources.

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein, et al., Nucl. Acids Res., 1988,16: 3209, methylphosphonate oligonucleotides may be prepared by use of controlled pore glass polymer supports (Sarin, et al., 1988, Proc. Nat. Acad. Sci. U.S.A. 85: 7448-7451), etc. In another embodiment, the oligonucleotide is a 2′-O-methylribonucleotide (Inoue, et al., Nucl. Acids Res., 1987, 15: 6131-6148), or a chimeric RNA-DNA analog (Inoue, et al., 1987, FEBS Lett., 215: 327-330).

Probes having sequences of genes listed in Tables I-IV may also be generated synthetically. Single-step assembly of a gene from large numbers of oligodeoxyribonucleotides may be done as described by Stemmer, et al., Gene (Amsterdam), 1995, 164(1):49-53. In this method, assembly PCR (the synthesis of long DNA sequences from large numbers of oligodeoxyribonucleotides (oligos)) is described. The method is derived from DNA shuffling (Stemmer, Nature, 1994, 370:389-391), and does not rely on DNA ligase, but instead relies on DNA polymerase to build increasingly longer DNA fragments during the assembly process. For example, a 1.1-kb fragment containing the TEM-1 beta-lactamase-encoding gene (bla) may be assembled in a single reaction from a total of 56 oligos, each 40 nucleotides (nt) in length. The synthetic gene may be PCR amplified and makes this approach a general method for the rapid and cost-effective synthesis of any gene.

“Rapid amplification of cDNA ends,” or RACE, is a PCR method that may be used for amplifying cDNAs from a number of different RNAs. The cDNAs may be ligated to an oligonucleotide linker and amplified by PCR using two primers. One primer may be based on sequence from the instant nucleic acids, for which full length sequence is desired, and a second primer may comprise a sequence that hybridizes to the oligonucleotide linker to amplify the cDNA. A description of this method is reported in PCT Pub. No. WO 97/19110.

In another embodiment, the invention provides a composition comprising a plurality of agents which may detect a polypeptide encoded by a gene from Tables I-IV. An agent may be, e.g., an antibody. Antibodies to polypeptides described herein may be obtained commercially, or they may be produced according to methods known in the art.

The probes may be attached to a solid support, such as paper, membranes, filters, chips, pins or glass slides, or any other appropriate substrate, such as those further described herein. For example, probes of genes from Tables I-IV may be attached covalently or non-covalently to membranes for use, e.g., in dotblots, or to solids such as to create arrays, e.g., microarrays.

6.5. Alternative Diagnostic Methods

In other embodiments, the assaying of the modulation of gene expression via can be performed using a Real Time-PCR assay. Total mRNA is extracted as described above and subjected to the reverse transcription using an RNA-directed DNA polymerase, such as reverse transcriptase isolated from AMV, MoMuLV or recombinantly produced. The cDNAs produced by the latter procedure can be amplified in the presence of Taq polymerase and the amplification monitored in an appropriate apparatus in real time as a function of PCR cycle number under the appropriate conditions that yield measurable signals, for example, in the presence of dyes that yield a particular absorbance reading when bound to duplex DNA. The relative concentrations of the mRNAs corresponding to chosen genes can be calculated from the cycle midpoints of their respective Real Time-PCR amplification curves and compared between cells of a subject treated with a candidate therapeutic relative to a control cell in order to determine the increase or decrease in mRNA levels in a quantitative fashion.

In other embodiments of the diagnostic methods contemplated by the present invention, the method of diagnosis comprises the steps of determining the level and/or activity of a protein encoded by a gene selected from Tables I-IV in the adipose cells of a subject undergoing treatment with a PPARγ ligand, and comparing the activity of said protein in said subject's cells before treatment with the PPARγ ligand. Assays to determine the activity of a particular protein are routinely used in the art, are well-known to one of skill in the art, and may be adapted to the methods of the present invention with no more than routine experimentation.

7. Pharmaceutical ComDositions of Therapeutic Agents

The therapeutic agents identified using the methods provided by the invention may be incorporated into a pharmaceutical composition, dispersed in a pharmaceutically-acceptable carrier, vehicle or diluent. In one embodiment, the pharmaceutical composition comprises a pharmaceutically-acceptable excipient. The compounds of the present invention may be administered by any suitable means, depending, for example, on their intended use, as is well known in the art, based on the present description. For example, if compounds of the present invention are to be administered orally, they may be formulated as tablets, capsules, granules, powders or syrups. Alternatively, formulations of the present invention may be administered parenterally as injections (intravenous, intramuscular or subcutaneous), drop infusion preparations or suppositories. For application by the ophthalmic mucous membrane route, compounds of the present invention may be formulated as eyedrops or eye ointments. These formulations may be prepared by conventional means, and, if desired, the compounds may be mixed with any conventional additive, such as an excipient, a binder, a disintegrating agent, a lubricant, a corrigent, a solubilizing agent, a suspension aid, an emulsifying agent or a coating agent.

In formulations of the subject invention, wetting agents, emulsifiers and lubricants, such as sodium lauryl sulfate and magnesium stearate, as well as coloring agents, release agents, coating agents, sweetening, flavoring and perfuming agents, preservatives and antioxidants may be present in the formulated agents.

Subject compounds may be suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of agent that may be combined with a carrier material to produce a single dose vary depending upon the subject being treated, and the particular mode of administration.

Methods of preparing these formulations can include the step of bringing into association agents of the present invention with the carrier, vehicle or diluent and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a compound thereof as an active ingredient. Compounds of the present invention may also be administered as a bolus, electuary, or paste.

In solid dosage forms for oral administration (capsules, tablets, pills, dragees, powders, granules and the like), the therapeutic agent is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the supplement or components thereof moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the compound, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to compounds, may contain suspending agents as, for example, ethoxylated isostearyl alcohols, polyoxyethylencoordinatione sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a therapeutic agent of the present invention with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent. Formulations which are suitable for vaginal administration also include pessaries, tampons, creams, gels, pastes, foams or spray formulations containing such carriers as are known in the art to be appropriate.

Dosage forms for transdermal administration of a supplement or component includes powders, sprays, ointments, pastes, creams, lotions, gels, solutions, patches and inhalants. The active component may be mixed under sterile conditions with a pharmaceutically acceptable carrier, and with any preservatives, buffers, or propellants which may be required. For transdermal administration of transition metal complexes, the complexes may include lipophilic and hydrophilic groups to achieve the desired water solubility and transport properties.

The ointments, pastes, creams and gels may contain, in addition to a supplement or components thereof, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a supplement or components thereof, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compounds of the present invention may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compound.

Ordinarily, an aqueous aerosol is made by formulating an aqueous solution or suspension of the compound together with conventional pharmaceutically acceptable carriers and stabilizers. The carriers and stabilizers vary with the requirements of the particular compound, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this invention suitable for parenteral administration comprise one or more components of a supplement in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

8. Methods of Treating a Disease Associated with a PPARγ Receptor

The pharmaceutical compositions of the present invention may be used in a variety of treatments of diseases. In one embodiment, a method of treatment comprises administering a therapeutically-effective amount of a pharmaceutical composition to said subject to modulate, i.e. to stimulate or inhibit, the expression of a gene or group of genes selected from the target genes of the invention. In another embodiment, a method for treatment comprises administering a therapeutically-effective amount of a pharmaceutical composition to said subject to inhibit or stimulate the activity of a protein or proteins encoded by one more genes selected from the target genes of the invention. In one embodiment of the present invention, methods of treating a subject comprises administering to said subject a protein encoded by one of the genes of Tables I-IV of the present invention.

As those skilled in the art will understand, the dosage of any agent, compound, drug, etc., of the present invention will vary depending on the symptoms, age and body weight of the patient, the nature and severity of the disorder to be treated or prevented, the route of administration, and the form of the supplement. Any of the subject formulations may be administered in any suitable dose, such as, for example, in a single dose or in divided doses. Dosages for the compounds of the present invention, alone or together with any other compound of the present invention, or in combination with any compound deemed useful for the particular disorder, disease or condition sought to be treated, may be readily determined by techniques known to those of skill in the art, based on the present description, and as taught herein. Also, the present invention provides mixtures of more than one subject compound, as well as other therapeutic agents.

The precise time of administration and amount of any particular compound that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more relevant indices at predetermined times during a 24-hour period. Treatment, including supplement, amounts, times of administration and formulation, may be optimized according to the results of such monitoring. The patient may be periodically reevaluated to determine the extent of improvement by measuring the same parameters, the first such reevaluation typically occurring at the end of four weeks from the onset of therapy, and subsequent reevaluations occurring every four to eight weeks during therapy and then every three months thereafter. Therapy may continue for several months or even years, with a minimum of one month being a typical length of therapy for humans. Adjustments to the amount(s) of agent administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The combined use of several compounds of the present invention, or alternatively other therapeutic agents, may reduce the required dosage for any individual component because the onset and duration of effect of the different components may be complimentary. In such combined therapy, the different active agents may be delivered together or separately, and simultaneously or at different times within the day.

9. Monitoring a Patient's ResDonse to the Therapeutic

The present invention further provides for methods of diagnosing a patient's response to treatment with a PPARγ ligand. Furthermore, the present invention provides prognostic methods for evaluating the progression of treatment with a PPARγ ligand. The invention provides panels of genes identified via gene expression profiling as being involved in the therapeutic response to treatment with PPARγ ligands. The genes, which are up- or down-regulated in response to treatment with PPARγ ligand are referred to herein as an “expression signature”. Accordingly, the expression signature of a cell containing the genes from Tables I-IV may be used diagnostically and prognostically for treatment of a PPARγ associated disease, such as Type II diabetes, with a PPARγ ligand. Exemplary diagnostic tools and assays are set forth below, under (i) to (iv), followed by exemplary methods for conducting these assays.

(i) In one embodiment, the invention provides a method for determining whether a subject is responsive to treatment with a PPARγ ligand. In a certain embodiment such a method comprises determining the levels of expression of one or more genes which are up-regulated in an adipose cell of the subject undergoing treatment with a PPARγ ligand and comparing these levels of expression with the levels of expression of the genes in an adipose cell of a subject not treated with the PPARγ ligand, or of the same subject before treatment with the PPARγ ligand, such that the up-regulation of one or more genes from Tables I or III is indicative that the subject is responsive to treatment with the PPARγ ligand. In a further embodiment such a method comprises determining the levels of expression of one or more genes which are down-regulated in an adipose cell of the subject undergoing treatment with PPARγ ligand and comparing these levels of expression with the levels of expression of the genes in an adipose cell of a subject not treated with the PPARγ ligand, or of the same subject before treatment with the PPARγ ligand, such that the down-regulation of one or more genes from Tables II or IV is indicative that the subject is responsive to treatment with the PPARγ ligand.

(ii) In another embodiment, the invention provides a method for determining whether a subject would be responsive to treatment with a PPARγ ligand. In a certain embodiment, such a method comprises determining the levels of expression of one or more genes from Tables I or III in an adipose cell after incubating the adipose cell with the PPARγ ligand, such that up-regulation of the one or more genes from Tables I or III is indicative that the subject would be responsive to treatment with the PPARγ ligand. In a further embodiment, such a method comprises determining the levels of expression of one or more genes from Table II or IV in an adipose cell after incubating the adipose cell with the PPARγ ligand, such that down-regulation of the one or more genes from Tables II or IV is indicative that the subject would be responsive to treatment with the PPARγ ligand.

(iii) The invention may also provide methods for selecting a therapy for a disease associated with PPARγ for a patient from a selection of several different treatments. Certain subjects may respond better to one type of PPARγ ligand over another. In a certain embodiment, the method comprises comparing the expression level of one or more genes from Tables I or III in an adipose cell obtained from a subject after incubating the adipose cell with a PPARγ ligand, and comparing these levels of expression with the levels of expression of the one or more genes in an adipose cell of the subject before incubation with the PPARγ ligand, and repeating the said comparing with at least one or more PPARγ ligands in order to select the ligand for which the treatment of said adipose cells results in the greatest number of up-regulated genes from Tables I and III In a further embodiment, the method comprises comparing the expression level of one or more genes from Tables II or IV in an adipose cell obtained from a subject after incubating the adipose cell with a PPARγ ligand, and comparing these levels of expression with the levels of expression of the one or more genes in an adipose cell of the subject before incubation with the PPARγ ligand, and repeating the said comparing with at least one or more PPARγ ligands in order to select the ligand for which the treatment of said adipose cells results in the greatest number of down-regulated genes from Tables II and IV. In another embodiment one dose of the PPARγ ligand could be administered before obtaining adipose cells from the subject and comparing the expression levels of one or more genes from Tables I or III in the adipose cell to the levels of expression of the one or more genes in an adipose cell of the subject before administration of the PPARγ ligand in order to determine whether the selected genes have been up-regulated in said subject. In a similar embodiment one dose of the PPARγ ligand could be administered before obtaining adipose cells from the subject and comparing the expression levels of one or more genes from Table II or III in the adipose cell to the levels of expression of the one or more genes in an adipose cell of the subject before administration of the PPARγ ligand in order to determine whether the selected genes have been down-regulated in said subject.

(iv) In yet another embodiment, the invention provides methods for determining the probability of occurrence of side effects (such as edema) in a subject in response to treatment with known ligands of the PPARγ receptor. As part of the latter embodiment such a method comprises determining the levels of expression of genes in an adipose cell of the subject undergoing treatment with PPARγ ligand and obtaining a ratio of the number of genes up-regulated by all ligands as listed in Table I to the total number of genes up-regulated by the particular ligand of said treatment where a certain ratio is indicative of a high probability of occurrence of the side effect. In a further embodiment such a method comprises determining the levels of expression of genes in an adipose cell of the subject undergoing treatment with PPARγ ligand and obtaining a ratio of the number of genes down-regulated by all ligands as listed in Table II to the total number of genes down-regulated by the particular ligand of said treatment where a certain ratio is indicative of a high probability of occurrence of the side effect.

A person of skill in the art will recognize that in certain diagnostic and prognostic assays, it will be sufficient to assess the level of expression of a single gene from Tables I-IV and that in others, the expression of two or more is preferred, whereas still in others, the expression of essentially all the genes from Tables I-IV is preferably assessed.

Set forth below are exemplary methods which may be used to determine the level of expression of one or more genes from Tables I-IV, e.g., for use in the above-described methods. For example, the level of expression of a gene may be determined by reverse transcription-polymerase chain reaction (RT-PCR); dotblot analysis; Northern blot analysis and in situ hybridization. In a preferred embodiment, the level of expression is determined by using a microarray which contains probes of the genes that are up- or down-regulated as listed in Tables I-IV. In another embodiment, the level of protein encoded by one or more of the genes that are up- or down-regulated as listed in Tables I-IV is determined in a cell of the type that is diseased. This may be done by a variety of methods, e.g., immunohistochemistry.

10. Kits

The invention further provides kits for determining the expression level of genes from Tables I-IV. The kits may be useful for identifying subjects that are responsive to treatment with a PPARγ ligand, as well as for identifying and validating therapeutics for a disease associated with the PPARγ receptor. In one embodiment, the kit comprises a computer readable medium on which is stored one or more gene expression profiles of adipose cells of a subject treated with a PPARγ ligand, or at least values representing levels of expression of one or more genes whose expression is characteristic of treatment with a PPARγ ligand. The computer readable medium may also comprise gene expression profiles of counterpart untreated adipose cells and any other gene expression profile described herein. The kit may comprise expression profile analysis software capable of being loaded into the memory of a computer system.

A kit may comprise appropriate reagents for determining the level of protein activity in the adipose cells of a subject.

A kit may comprise a microarray comprising probes of genes from Tables I-IV. A kit may comprise one or more probes or primers for detecting the expression level of one or more genes from Tables I-IV and/or a solid support on which probes attached and which may be used for detecting expression of one or more genes whose expression is characteristic of treatment with a PPARγ ligand. A kit may further comprise nucleic acid controls, buffers, and instructions for use.

Kit components may be packaged for either manual or partially or wholly automated practice of the foregoing methods. In other embodiments involving kits, this invention provides a kit including compositions of the present invention, and optionally instructions for their use. Such kits may have a variety of uses, including, for example, imaging, diagnosis, therapy, and other applications.

11. Therapeutic Methods

11.1. Methods for Increasing the Expression of a Protein in Cells of a Patient

If it is shown that gene Y from Table I or Table III is important in a disease associated with PPARγ, and that the disease can be treated by increasing the level of polypeptide Y in the diseased cells, the following methods of treatment of the disease are available.

(i) Administration of a Nucleic Acid Encoding Polypeptide Y to a Subject

In one embodiment, a nucleic acid encoding polypeptide Y, or an equivalent thereof, such as a functionally active fragment of polypeptide Y, is administered to a subject, such that the nucleic acid arrives at the site of the diseased cells, traverses the cell membrane and is expressed in the diseased cell.

Determining which portion of the polypeptide is sufficient for improving the disease associated with PPARγ or which polypeptides derived from polypeptide Y are “equivalents” which can be used for treating the disease, can be done in in vitro assays. For example, expression plasmids encoding various portions of the polypeptide can be transfected into cells, e.g., diseased cells of the disease, and the effect of the expression of the portion of the polypeptide in the cells can be determined, e.g., by visual inspection of the phenotype of the cell or by obtaining the expression profile of the cell, as further described herein.

Any means for the introduction of polynucleotides into mammals, human or non-human, may be adapted to the practice of this invention for the delivery of the various constructs of the invention into the intended recipient. In one embodiment of the invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See, e.g., Canonico, et al, Am J Respir Cell Mol Biol, 10:24-29, 1994; Tsan, et al, Am J Physiol, 268; Alton, et al., Nat Genet., 1993, 5:135-142 and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject.

In a preferred method of the invention, the DNA constructs are delivered using viral vectors. The transgene may be incorporated into any of a variety of viral vectors useful in gene therapy, such as recombinant retroviruses, adenovirus, adeno-associated virus (AAV), and herpes simplex virus-1, or recombinant bacterial or eukaryotic plasmids. While various viral vectors may be used in the practice of this invention, AAV- and adenovirus-based approaches are of particular interest. Such vectors are generally understood to be the recombinant gene delivery system of choice for the transfer of exogenous genes in vivo, particularly into humans. The following additional guidance on the choice and use of viral vectors may be helpful to the practitioner. As described in greater detail below, such embodiments of the subject expression constructs are specifically contemplated for use in various in vivo and ex vivo gene therapy protocols.

(ii) Administration of a Polypeptide Y to a Subject

In another embodiment, polypeptide Y, or an equivalent thereof, e.g., a functional fragment thereof, is administered to the subject such that it reaches the diseased cells of a disease associated with PPARγ, and traverses the cellular membrane. Polypeptides can be synthesized in prokaryotes or eukaryotes or cells thereof and purified according to methods known in the art. For example, recombinant polypeptides can be synthesized in human cells, mouse cells, rat cells, insect cells, yeast cells, and plant cells. Polypeptides can also be synthesized in cell free extracts, e.g., reticulocyte lysates or wheat germ extracts. Purification of proteins can be done by various methods, e.g., chromatographic methods (see, e.g., Scopes, Robert K., Protein Purification: Principles and Practice, Third Ed. Springer-Verlag, N.Y. 1994). In one embodiment, the polypeptide is produced as a fusion polypeptide comprising an epitope tag consisting of about six consecutive histidine residues. The fusion polypeptide can then be purified on a Ni⁺⁺ column. By inserting a protease site between the tag and the polypeptide, the tag can be removed after purification of the peptide on the Ni⁺⁺ column. These methods are well known in the art and commercial vectors and affinity matrices are commercially available.

Administration of polypeptides can be done by mixing them with liposomes, as described above. The surface of the liposomes can be modified by adding molecules that will target the liposome to the desired physiological location.

In one embodiment, polypeptide Y is modified so that its rate of traversing the cellular membrane is increased. For example, the polypeptide can be fused to a second peptide which promotes “transcytosis,” e.g., uptake of the peptide by cells. In one embodiment, the peptide is a portion of the HIV transactivator (TAT) protein, such as the fragment corresponding to residues 37-62 or 48-60 of TAT, portions which are rapidly taken up by cell in vitro (Green and Loewenstein, Cell, 1989, 55:1179-1188). In another embodiment, the internalizing peptide is derived from the Drosophila antennapedia protein, or homologs thereof. The 60 amino acid long homeodomain of the homeo-protein antennapedia has been demonstrated to translocate through biological membranes and can facilitate the translocation of heterologous polypeptides to which it is couples. Thus, polypeptides can be fused to a peptide consisting of about amino acids 42-58 of Drosophila antennapedia or shorter fragments for transcytosis. See for example Derossi, et al., J Biol Chem, 1996, 271:18188-18193; Derossi, et al., J Biol Chem, 1994, 269:10444-10450; and Perez, et al., J Cell Sci, 1992, 102:717-722.

(iii) Use of Agents Stimulating Transcription or Polypeptide Activity

In another embodiment, a pharmaceutical composition comprising a compound that stimulates the level of expression of gene Y or the activity of polypeptide Y in a cell is administered to a subject, such that the level of expression of gene Y in the diseased cells is increased or even restored, and disease Y is improving in the subject. Alternatively, such compounds can be designed or identified according to methods known in the art and the methods disclosed herein.

11.2. Methods for Reducing Expression of Gene X in the Cells of a Patient

If it is shown that gene X from Table II or Table IV is important in a disease associated with PPARγ, and that the disease can be treated by decreasing the level of polypeptide X in the diseased cells, the following methods of treatment of the disease are available.

(i) Antisense Nucleic Acids

One method for decreasing the level of expression of a gene is to introduce into the cell antisense molecules which are complementary to at least a portion of gene X or RNA of gene X. An “antisense” nucleic acid as used herein refers to a nucleic acid capable of hybridizing to a sequence-specific (e.g., non-poly A) portion of the target RNA, for example its translation initiation region, by virtue of some sequence complementarity to a coding and/or non-coding region. The antisense nucleic acids of the invention can be oligonucleotides that are double-stranded or single-stranded, RNA or DNA or a modification or derivative thereof, which can be directly administered in a controllable manner to a cell or which can be produced intracellularly by transcription of exogenous, introduced sequences in controllable quantities sufficient to perturb translation of the target RNA.

Preferably, antisense nucleic acids are of at least six nucleotides and are preferably oligonucleotides (ranging from 6 to about 200 oligonucleotides). In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof, single-stranded or double-stranded. The oligonucleotide can be modified at the base moiety, sugar moiety, or phosphate backbone. The oligonucleotide may include other appending groups such as peptides, or agents facilitating transport across the cell membrane (see, e.g., Letsinger, et al., Proc. Natl. Acad. Sci. U.S.A., 1989, 86: 6553-6556; Lemaitre, et al., Proc. Natl. Acad. Sci., 1987, 84: 648-652: PCT Publication No. WO 88/09810, published Dec. 15, 1988), hybridization-triggered cleavage agents (see, e.g., Krol et al., BioTechniques, 1988, 6: 958-976) or intercalating agents (see, e.g., Zon, Pharm. Res., 1988, 5: 539-549).

In a preferred aspect of the invention, an antisense oligonucleotide is provided, preferably as single-stranded DNA. The oligonucleotide may be modified at any position on its structure with constituents generally known in the art. For example, the antisense oligonucleotides may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

In another embodiment, the oligonucleotide comprises at least one modified sugar moiety selected from the group including, but not limited to, arabinose, 2-fluoroarabinose, xylulose, and hexose.

In yet another embodiment, the oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the oligonucleotide is a 2-a-anomeric oligonucleotide. An α-anomeric oligonucleotide forms specific double-stranded hybrids with complementary RNA in which, contrary to the usual β-units, the strands run parallel to each other (Gautier, et al., Nucl. Acids Res., 1987, 15:6625-6641).

The oligonucleotide may be conjugated to another molecule, e.g., a peptide, hybridization triggered cross-linking agent transport agent, hybridization-triggered cleavage agent, etc. An antisense molecule can be a “peptide nucleic acid” (PNA). PNA refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

The antisense nucleic acids of the invention comprise a sequence complementary to at least a portion of a target RNA species. However, absolute complementarity, although preferred, is not required. A sequence “complementary to at least a portion of an RNA,” as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a target RNA it may contain and still form a stable duplex (or triplex, as the case may be). One skilled in the art can ascertain a tolerable degree of mismatch by use of standard procedures to determine the melting point of the hybridized complex. The amount of antisense nucleic acid that will be effective in the inhibiting translation of the target RNA can be determined by standard assay techniques.

The synthesized antisense oligonucleotides can then be administered to a cell in a controlled manner. For example, the antisense oligonucleotides can be placed in the growth environment of the cell at controlled levels where they may be taken up by the cell. The uptake of the antisense oligonucleotides can be assisted by use of methods well known in the art.

In an alternative embodiment, the antisense nucleic acids of the invention are controllably expressed intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequences encoding the antisense RNAs can be by any promoter known in the art to act in a cell of interest. Such promoters can be inducible or constitutive. Most preferably, promoters are controllable or inducible by the administration of an exogenous moiety in order to achieve controlled expression of the antisense oligonucleotide. Such controllable promoters include the Tet promoter. Other usable promoters for mammalian cells include, but are not limited to: the SV40 early promoter region (Bernoist and Chambon, 1981, Nature 290: 304-310), the promoter contained in the 3′ long terminal repeat of Rous sarcoma virus (Yamamoto, et al., Cell, 1980, 22: 787-797), the herpes thymidine kinase promoter (Wagner et al., 1981, Proc. Natl. Acad. Sci. U.S.A. 78: 1441-1445), the regulatory sequences of the metallothionein gene (Brinster, et al., Nature, 1982, 296: 39-42), etc.

Antisense therapy for a variety of cancers is in clinical phase and has been discussed extensively in the literature. Reed reviewed antisense therapy directed at the Bcl-2 gene in tumors; gene transfer-mediated overexpression of Bcl-2 in tumor cell lines conferred resistance to many types of cancer drugs. (Reed, J. C., N.C.I. (1997) 89:988-990). The potential for clinical development of antisense inhibitors of ras is discussed by Cowsert, L. M., Anti-Cancer Drug Design, 1997, 12:359-371. Additional important antisense targets include leukemia (Geurtz, A. M., Anti-Cancer Drug Design, 1997, 12:341-358); human C-ref kinase (Monia, B. P., Anti-Cancer Drug Design, 1997, 12:327-339); and protein kinase C (McGraw et al., Anti-Cancer Drug Design, 1997, 12:315-326.

(ii) Ribozymes

In another embodiment, the level of a particular mRNA or polypeptide in a cell is reduced by introduction of a ribozyme into the cell or nucleic acid encoding such. Ribozyme molecules designed to catalytically cleave mRNA transcripts can also be introduced into, or expressed, in cells to inhibit expression of gene Y (see, e.g., Sarver, et al., 1990, Science 247:1222-1225 and U.S. Pat. No. 5,093,246). One commonly used ribozyme motif is the hammerhead, for which the substrate sequence requirements are minimal. Design of the hammerhead ribozyme is disclosed in Usman, et al., Current Opin. Struct. Biol., 1996, 6:527-533. Usman also discusses the therapeutic uses of ribozymes. Ribozymes can also be prepared and used as described in Long, et al., FASEB J., 1993, 7:25; Symons, Ann. Rev. Biochem., 1992, 61:641; Perrotta, et al., Biochem., 1992, 31:16-17; Ojwang, et al., Proc. Natl. Acad. Sci. (USA), 1992, 89:10802-10806; and U.S. Pat. No. 5,254,678. Ribozyme cleavage of HIV-I RNA is described in U.S. Pat. No. 5,144,019; methods of cleaving RNA using ribozymes is described in U.S. Pat. No. 5,116,742; and methods for increasing the specificity of ribozymes are described in U.S. Pat. No. 5,225,337 and Koizumi et al., Nucleic Acid Res., 1989, 17:7059-7071. Preparation and use of ribozyme fragments in a hammerhead structure are also described by Koizumi et al., Nucleic Acids Res., 1989, 17:7059-7071. Preparation and use of ribozyme fragments in a hairpin structure are described by Chowrira and Burke, Nucleic Acids Res., 1992, 20:2835. Ribozymes can also be made by rolling transcription as described in Daubendiek and Kool, Nat. Biotechnol., 1997, 15(3):273-277.

(iii) siRNAs

Another method for decreasing or blocking gene expression is by introducing double stranded small interfering RNAs (siRNAs), which mediate sequence specific mRNA degradation. RNA interference (RNAi) is the process of sequence-specific, post-transcriptional gene silencing in animals and plants, initiated by double-stranded RNA (dsRNA) that is homologous in sequence to the silenced gene. In vivo, long dsRNA is cleaved by ribonuclease III to generate 21- and 22-nucleotide siRNAs. It has been shown that 21-nucleotide siRNA duplexes specifically suppress expression of endogenous and heterologous genes in different mammalian cell lines, including human embryonic kidney (293) and HeLa cells (Elbashir, et al., Nature, 2001 ;411(6836):494-8).

(iv) Triplex Formation

Gene expression can be reduced by targeting deoxyribonucleotide sequences complementary to the regulatory region of the target gene (i.e., the gene promoter and/or enhancers) to form triple helical structures that prevent transcription of the gene in target cells in the body. (See generally, Helene, C., Anticancer Drug Des., 6(6):569-84; Helene, C., et al., 1992, Ann, N.Y. Accad. Sci., 660:27-36; and Maher, L. J., Bioassays, 199214(12):807-15).

(v) Aptamers

In a further embodiment, RNA aptamers can be introduced into or expressed in a cell. RNA aptamers are specific RNA ligands for proteins, such as for Tat and Rev RNA (Good et al., Gene Therapy, 1997, 4: 45-54) that can specifically inhibit their translation.

(vi) Dominant Negative Mutants

Another method of decreasing the biological activity of a polypeptide is by introducing into the cell a dominant negative mutant. A dominant negative mutant polypeptide will interact with a molecule with which the polypeptide normally interacts, thereby competing for the molecule, but since it is biologically inactive, it will inhibit the biological activity of the polypeptide. A dominant negative mutant can be created by mutating the substrate-binding domain, the catalytic domain, or a cellular localization domain of the polypeptide. Preferably, the mutant polypeptide will be overproduced. Point mutations are made that have such an effect. In addition, fusion of different polypeptides of various lengths to the terminus of a protein can yield dominant negative mutants. General strategies are available for making dominant negative mutants. See Herskowitz, Nature, 1987, 329:219-222.

(vi) Use of Agents Inhibiting Transcription or Polypeptide Activity

In another embodiment, a compound decreasing the expression of gene X or the activity of polypeptide X is administered to a subject having disease D, such that the level of polypeptide X in the diseased cells decreases, and the disease is improved. Additional compounds can be identified as further described herein.

5. Exemplification

The present invention is further illustrated by the following examples which should not be construed as limiting in any way. The contents of all cited references including literature references, issued patents, published or non published patent applications as cited throughout this application are hereby expressly incorporated by reference in their entireties. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Molecular Cloning A Laboratory Manual, 2nd Ed., ed. by Sambrook, Fritsch and Maniatis (Cold Spring Harbor Laboratory Press: 1989); DNA Cloning, Volumes 1 and 11 (D. N. Glover ed., 1985); Oligonucleotide Synthesis (M. J. Gait ed., 1984); Mullis, et al. U.S. Pat. No. 4,683,195; Nucleic Acid Hybridization (B. D. Hames & S. J. Higgins eds. 1984); Transcription And Translation (B. D. Hames & S. J. Higgins eds. 1984); (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Vols. 154 and 155 (Wu et al. eds.), Immunochemical Methods In Cell And Molecular Biology (Mayer and Walker, eds., Academic Press, London, 1987); Handbook Of ExDerimental Immunology, Volumes I-IV (D. M. Weir and C. C. Blackwell, eds., 1986) (Cold Spring Harbor Laboratory Press, Cold Spring Harbor, N.Y., 1986).

### Example 1

### Genes that are Up- or Down-Regulated in Adipocytes Treated with a PPARγ Ligand

This example describes the identification of genes that are up- or down-regulated in 3T3-L1 adipocytes treated with a PPARγ ligand. 3T3-L1 cells were grown in the presence of 500 μM isobutylmethylxanthine (Sigma), 250 nM dexamthasone (Sigma), 1 μg/ml insulin (Sigma) and 10% fetal bovine serum (Hyclone) for 48 hours followed by an additional 48 hours in insulin and serum containing medium, and then maintained in serum only containing medium to obtain 3T3-L1 adipocytes. Twelve days after the beginning of the incubation with this inducer, which corresponds to 14 days post confluence, the 3T3-L1 adipocytes were incubated for 24 hours with one of the following PPARγ ligands or with vehicle (0.1% DMSO): 20 μM Troglitazone (Tro), 20 μM Pioglitazone (Pio), 20 μM MCC-555, 1 μM Rosiglitazone (Rosi), 1 μM Darglitazone (Dar), or 1 μM Farglitazar (FAR) (all synthesized at Pfizer). Following the incubation, RNA was extracted from the cells using Trizol (Invitrogen) followed by cleanup using an RNeasy kit (Qiagen) including DNase I treatment. Double stranded cDNA was synthesized with the SuperScript Choice system (Invitrogen) using a T7-(dT)₂₄ oligomer (Ambion) from the resulting RNA from each of these cell populations. Biotin labeled probes from the cDNAs were subsequently obtained by in vitro transcription using the BioArray High Yield RNA Transcript Labeling Kit (Enzo).

The probes were then hybridized to U74Aver2 Affymetrix gene chips. The chips were hybridized for 16 hours at 45° C. and 60 RPM in a rotisserie box. Statistical analysis was conducted with GeneSpring software (Silicon Genetics) and error analysis was conducted using the Global Error Model using the Benjamini and Hochberg false discovery rate multiple testing correction.

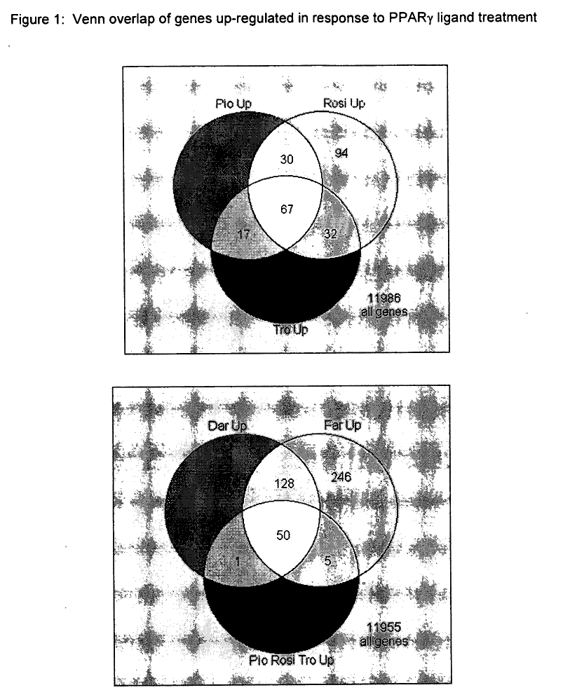

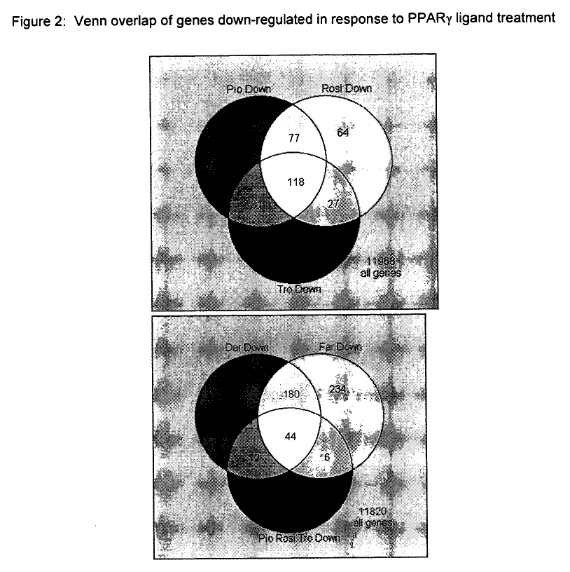

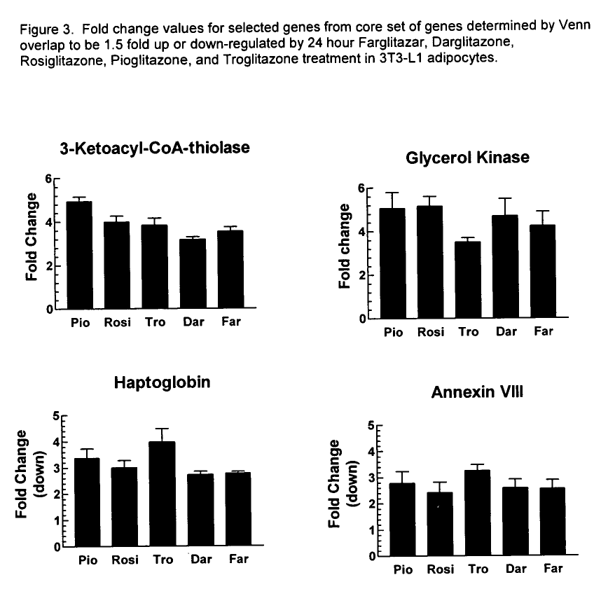

Genes which showed at least a 1.5 fold increase or decrease in expression in response to treatment with a PPARγ ligand were identified by Venn overlap. The total number of genes that were up-regulated in response to any of the PPARγ ligands tested was 970 and the total number of genes that were down-regulated in response to any of the PPARγ ligands tested was 1,072. Interestingly, 50 genes were up-regulated (FIG. 1) by each of the PPARγ ligands tested and 44 genes were down-regulated (FIG. 2) by each of the PPARγ ligands. The identities of these 50 and 44 genes are set forth in Tables I and II, respectively. Fold change values for 4 representative genes selected from the list of genes that were up or down-regulated in response to any of the PPARγ ligands tested are shown in FIG. 3.

A complementary statistical analysis, based on the concept of a sliding fold cutoff, was performed on the data which produced an additional 23 genes significantly up-regulated and an additional 24 genes significantly down-regulated in response to all of the PPARγ ligands tested. The identities of these 23 and 24 genes are set forth in Tables III and IV, respectively. This analysis employs a method based on that described by Novak, et al (Genomics, 2002, 79:104) where genes with similar expression levels are binned together and an artificial standard deviation is determined for each bin. The fold change for each gene is determined and this value is expressed relative to the standard deviation based on expression level, thus giving a Z score. This Z score is then used to determine significance values.

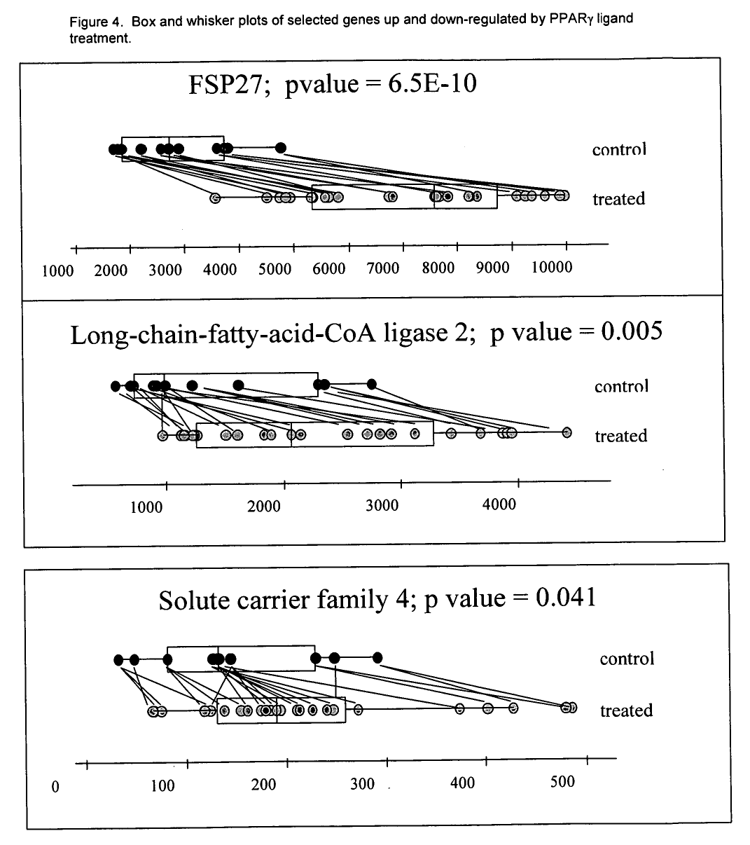

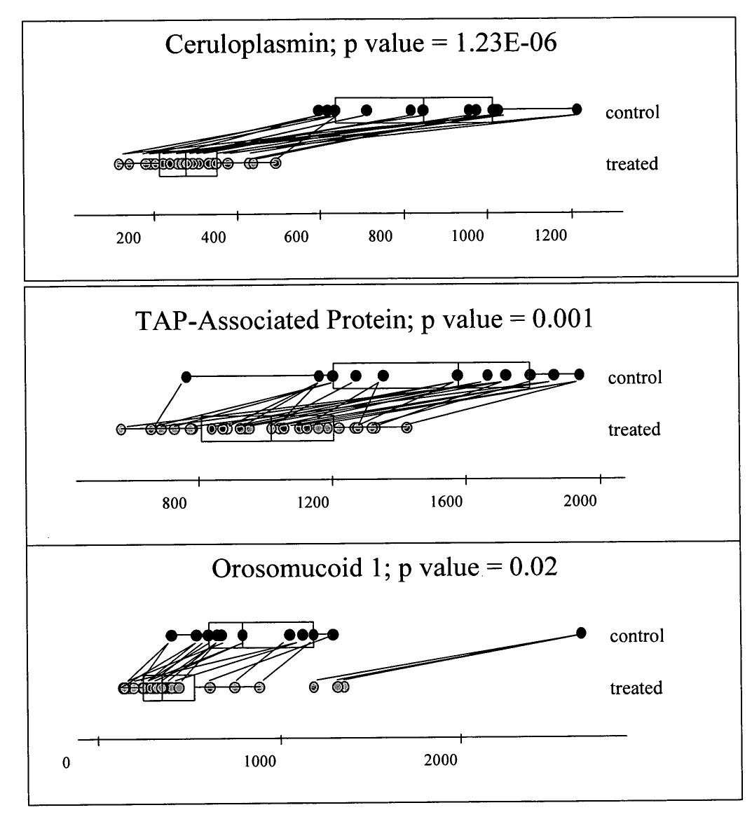

Box and whisker plot of identified genes are represented in FIG. 4. These plots illustrate the signal intensity from PPARγ ligand treated samples relative to the signal intensity from vehicle treated control samples. Lines connect treated samples and control samples analyzed in the same experiment (paired). The genes in these plots were selected at random to illustrate the relative signals from highly significant genes to borderline significant genes.

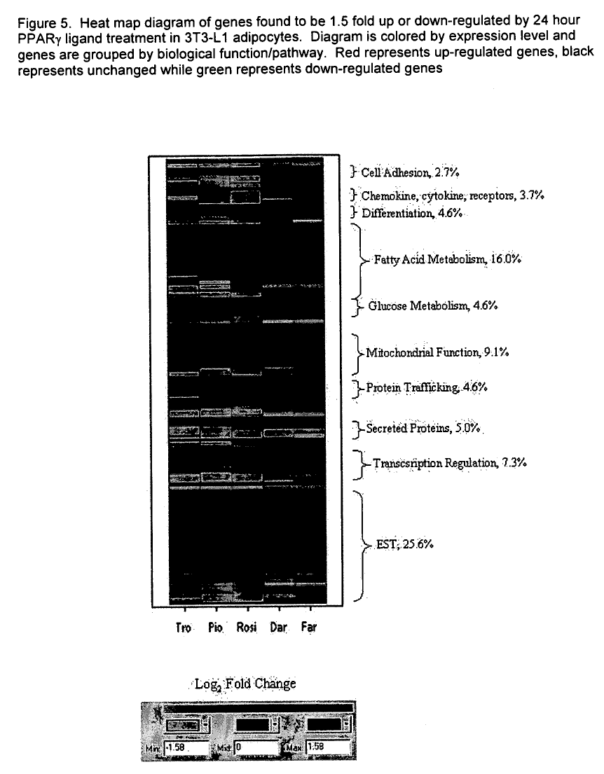

The identified core set of genes was grouped by biological pathway or function and a heat map was generated (Spotfire DecisionSite 6.2) showing relative expression levels of each gene for each ligand. FIG. 5 represents the overlapping core gene set grouped by function and expression level. The figure is a continuous color map with light green representing highly down regulated genes (3 fold down regulated), black represents genes unchanged relative to control, and light red representing genes highly up-regulated (3 fold up-regulated).

Within the set of genes identified as up or down-regulated by treatment with all PPARγ ligands tested are several genes that have been shown by our lab or others to potentially play a role in diabetes and/or insulin sensitization. The following genes are of particular interest and support the validity of the identified PPARγ efficacy signature:

PPARgamma

The PPARγ receptor is the known target for insulin sensitizing thiazoladinedione anti-diabetic agents. Therefore, a new method for activating this receptor has potential therapeutic benefits in all insulin resistant diseases such as Type 2 diabetes and others. Furthermore, the expression level of PPARγ itself, may also be important, since results from our laboratory have shown that a 50% decrease in PPARγ gene dosage in all tissues of the body leads to enhanced insulin sensitivity, whereas complete deletion of PPARγ receptor from skeletal muscle alone leads to insulin resistance. (Miles, et al., J. Clin. Invest., 2000, 105:287-292.)

Annexin II

Recent data from our lab indicate that annexin II protein levels are up-regulated by TZD treatment and that annexin II plays a positive role in insulin-stimulated GLUT4 translocation and glucose transport. It is reported in the literature that annexin II mRNA levels increase in adipocytes from diabetic rats treated with an insulin-sensitizing, non-TZD PPARgamma ligand. (Huang, et al., Diabetes, 2002, 51 (Suppl. 2):A292. Way, et al., Endocrinology, 2001,142:1269-1277.)

RGS2

RGS2 has been shown to dampen insulin action in published data from our lab which reports a potent negative regulatory effect of RGS2 protein on insulin stimulated GLUT4 translocation in 3T3-L1 adipocytes. (Imamura, et al, Mol. Cell. Biol., 1999, 19:6765-6774.)

PGC-1:

PPARgamma coactivator-1 has been shown to be involved with UCP expression and thermogenesis. Markedly reduced in ob/ob and db/db mice, and fa/fa rats. Polymorphisms have been linked to diabetes. Exercise upregulates PGC1 expression (muscle). PGC-1 over expression increases insulin sensitivity in adipocytes. Spiegelman and colleagues have published evidence in support of an insulin-sensitizing role for PGC-1 involving PPARγ Further, two recent publications show that polymorphic alleles of PGC-1 are associated with obesity and type 2 diabetes. (Puigserver, et al., Science, 1999, 286:1368-1371. Michael, et al., 2001, Proc. Natl. Acad. Sci. USA, 98:3820-3825. Esterbauer, et al., 2002, Diabetes 51:1281-1286. Hara, et al., Diabetologia, 2002, 45:740-743.)

C200rf24-Putative Rab5 Interacting Protein

We have published studies demonstrating a Rab5-dependent pathway that regulates GLUT4 distribution to the cell surface of adipocytes and that this pathway is directly regulated by insulin in a PI 3-kinase dependent manner. C20orf24, as a putative Rab5-interacting protein, may play a role in this important function of Rab5 in mediating insulin-stimulated cell surface GLUT4 levels. (Huang, et al., Proc Natl Acad Sci USA, 2001, 98(23):13084-9.)

CAP (c-Cbl-Associated Protein)

The protein CAP (c-Cbl-associated protein) has been shown to participate in insulin-mediated signaling and glucose transport. Thiazolidinedione treatment of 3T3-L1 adipocytes and Zucker lean and diabetic rats increases CAP protein levels and the CAP gene contains an active PPARgamma response element in its promoter. A polymorphism in the human homolog of CAP (SORBS1) associated with reduced incidence of obesity and type 2 diabetes. (Baumann, et al, J. Biol. Chem., 2001, 276(9):6065-8. Ribon, et al, Proc. Natl. Acad. Sci. USA, 1998, 95:14751-14756. Lin, et al, Hum Mol Genet., 2001, 10(17):1753-60.)

PEPCK

PEPCK is a key enzyme regulating glyceroneogenesis in adipocytes and, thus, free fafty acid re-esterification. Over-expression of PEPCK in mice leads to a reduction in circulating FFAs and increased adiposity without any detrimental effect on insulin sensitivity. PEPCK has a known responsive element in its promoter and PEPCK expression is upregulated in PPARγ ligand treated diabetic rats. (Franckhauser, et al, Diabetes, 2002, 51:624-630.)

Orosomucoid (Alpha-1-Acid Glycoprotein 1)

Orosomucoid presence in urine of type 11 diabetics is a significant predictive factor of patient mortality. (Like haptoglobin and ceruloplasmin, orosomucoid is also associated with inflammation.) Elevated in ob/ob liver, decreased in ob/ob WAT. (Christiansen, et al., Diabetologia, 2002, 45:115-120.)

Angiopoletin-Like 4:

Angiopoietin-like 4 is a secreted factor that is also known as PPARgamma angiopoietin related gene (PGAR). PGAR is a PPARgamma target gene that encodes an angiopoietin-like secreted glycoprotein. PGAR expression is highly adipose tissue selective (white and brown fat) over other tissues, PPARgamma ligands (Pio) upregulate PGAR expression and protein within 2 hours in NIH3T3 cells. PGAR levels increase dramatically during 3T3-L1 adipocyte differentiation. PGAR adipose gene expression is highly upregulated in mouse models of obesity (ob/ob) and diabetes (db/db) versus their lean controls. PGAR expression is increased in mice after short term fast (12 Hr) and is reversed upon refeeding. Leptin administration induced dietary restriction does not result in the upregulation of PGAR expression observed in pair-fed control mice. PGAR may be involved in adipocyte differentiation, could potentially be involved in metabolic homeostasis, or act upon liver or muscle to influence systemic insulin sensitivity and glucose metabolism. (Yoon, J C, et al., Mol Cell Biol, 2002, 20(14): 5343-49.)

Peroxisomal 3-Ketoacyl-CoA Thiolase & Sterol Carrier Protein 2

Both of these enzymes are involved in fatty acid beta-oxidation. They may reduce FFA secretion. This may reduce the accumulation of synthesized triglycerides within cells (particularly liver and skeletal muscle) which may lead to improved insulin sensitivity. The promoter of the peroxisomal 3-ketoacyl-CoA thiolase gene contains a functional PPAR responsive element. (Latruffe, et al., Biochem. Soc. Trans.,2001, 29:305-309.)

Estrogen Related Receptor Alpha

Induces transcription of key fatty acid beta oxidation enzyme (medium chain acyl CoA dehydrogenase) in adipose tissue. Activity may reduce FFA secretion and improve insulin sensitivity Estrogen related receptor alpha has recently been shown to interact with and modulate the activity of the transcriptional coactivator PGC-1. (Ichida, et al, J. Biol. Chem. Oct 22 [epub ahead of print].)

Haptoglobin

Plasma glycoprotein induced by cytokines and inflammation. Haptoglobin is significantly upregulated in WAT in ob/ob, db/db, and KKay mice, and plasma haptoglobin levels significantly higher in obese humans relative to lean controls. TNFa stimulates haptoglobin expression in WAT in vivo. (Chiellini, C., et al., J Cell Physiology, 2002,190:251-58.)

Pre B-Cell Leukemia Transcription Factor I

PBX1 is a member of the three-amino acid loop extension class of homeodomain transcription factors. PBX1^{+/-} mice show that PBX1 is required for pancreatic insulin secretion and that germline PBX1 inactivation led to inadequate levels of circulating insulin and impaired glucose tolerance. Reduction in PBX-1 may promote susceptibility to diabetes. (Kim, S. K., et al., Nat Gen, 2002, 30(4):430-35.)

Ribosomal Protein S6 Kinase

RSK2 (map kinase activated protein kinase). RSK2 is phosphorylated and activated by insulin stimulation in vivo. Basal and stimulated RSK2 activity is reduced in obese fa/fa rats compared to lean littermates. Exercise training significantly increases RSK2 activity in obese fa/fa rats. RSK2 activity is decreased in glucosamine induced insulin resistance in 3T3-L1 adipocytes. (Osman, A. A., et al., J Appl Physiol, 2001,90:454-60.)

Glycerol Kinase

Upregulation of glycerol kinase in adipose tissue induces futile cycle of triglyceride breakdown and resynthesis from glycerol and free fatty acid. Reduces free fatty acid secretion and thus maybe improves muscle insulin sensitivity.

Stearoyl CoA Desaturase

SCD-1 is the rate limiting enzyme in fatty acid biosynthesis. High SCD-1 activity is associated with obesity, diabetes, and atherosclerosis. Knockout mice have reduced adipocity, increased insulin sensitivity, and are resistant to diet induced weight gain. (Ntambi, et al. PNAS, 2002, 99(17):11482-86).

Resistin

Resistin is an dipocye secreted factor shown to reduce insulin sensitivity and is upregulated in obese mice. Resistin may link obesity and diabetes. (Hartman, et al., J. Biol. Chem., 2002, 277(22):19754-61.)

TABLE I
Identity of genes that are
up-regulated in response to PPARγ ligands

| AffyID | Genbank | p value | GeneName |
|---|---|---|---|
| 102114_f_at | gb\|AI326963 | 1.07E−18 | Angiopoietin-like 4 |
| 102016_at | M61737 | 5.45E−18 | FSP27; fat specific |
| 96119_s_at | NP_065606 | 1.08E−16 | Angiopoietin-like 4 |
| 96913_at | gb\|AW122615 | 1.70E−15 | similar to 3-ketoacyl CoA |
| 96841_at | P58750 | 1.34E−14 | Serine/threonine-protein |
| | | | kinase |
| 99571_at | gb\|AW012588 | 1.88E−14 | Peroxisomal 3-oxoacyl- |
| | | | CoA- |
| 102049_at | NP_038771 | 6.56E−14 | Pyruvate dehydrogenase |
| 102052_at | NP_080455 | 1.09E−13 | similar to CGI-58; |
| | | | Chanarin- |
| 160320_at | NP_033192 | 2.76E−12 | c-Cbl-associated protein; |
| | | | CAP |
| 103964_at | NP_031979 | 3.53E−12 | ESRRA; Estrogen-related |
| 101515_at | NP_056544 | 3.83E−12 | Acyl-coA oxidase 1 |
| | | | (EC 1.3.3.6) |
| 96134_at | gb\|AA755260 | 3.83E−12 | EST |
| 93360_at | O35621 | 8.87E−12 | Phosphomannomutase 1 |
| | | | (EC |
| 93051_at | NP_031966 | 1.05E−11 | Soluble epoxide hydrolase |
| | | | (EC |
| 96122_at | gb\|AW049373 | 1.15E−11 | EST |
| 95695_at | NP_065266 | 1.25E−11 | mCAC; carnitine/ |
| | | | acylcarnitine |
| 101525_at | gb\|AI848871 | 1.62E−11 | NADH dehydrogenase |
| 160107_at | NP_038584 | 7.40E−11 | HPRT; Hypoxanthine- |
| | | | guanine |
| | | | phosphoribosyltransferase |
| | | | (EC 2.4.2.8) |
| 97525_at | NP_032220 | 1.18E−10 | Glycerol kinase |
| | | | (EC 2.7.1.30) |
| 94276_at | NP_062631 | 1.29E−10 | KIK 1; Hydroxysteroid |
| | | | (17-beta) |
| | | | dehydrogenase |
| | | | (EC 1.1.1.-). |
| 94507_at | NP_032007 | 1.29E−10 | Long-chain-fatty- |
| | | | acid —CoA |
| | | | ligase 2 (EC 6.2.1.3) |
| 96090_g_at | gb\|AI255972 | 2.75E−10 | EST |
| 98589_at | NP_031434 | 3.33E−10 | Adipophilin (Adipose |
| | | | differentiation-related |
| | | | protein) (ADRP). |
| 99535_at | O35710 | 4.92E−10 | Nocturnin (CCR4 protein |
| | | | homolog). |
| 100464_at | gb\|AI840585 | 4.92E−10 | EST |
| 100569_at | NP_031611 | 1.33E−09 | Annexin II (Lipocortin II) |
| | | | (Calpactin I heavy chain) |
| 95787_s_at | P32020 | 1.33E−09 | Sterol carrier protein 2; |
| | | | SCPX |
| 93270_at | NP_083060 | 1 .33E−09 | Coagulation factor XIIa |
| | | | homolog |
| 95026_at | gb\|AW047688 | 3.33E−09 | EST |
| 160333_at | NP_080400 | 6.23E−09 | C20orf24; putative Rab5 |
| | | | interacting protein |
| 94325_at | AW124932 | 1.63E−08 | Pbx1; pre B-cell leukemia |
| | | | transcription factor 1 |
| 97429_at | gb\|AW048113 | 9.70E−08 | EST |
| 98457_at | NP_061230 | 9.70E−08 | Solute carrier family 4 |
| | | | (anion exchanger), |
| | | | member 4 |
| 92805_s_at | NP_031513 | 1.37E−07 | ADP-ribosylation factor- |
| | | | like protein 4 (ARL4) |
| 96900_at | gb\|AW125480 | 3.08E−07 | EST |
| 95523_at | gb\|AI839718 | 1.01E−06 | microsomal signal |
| | | | peptidase 23 kDa subunit |
| 160737_at | gb\|AW060927 | 1.14E−06 | Similar to Lanosterol |
| | | | synthathase |
| 92715_at | AL078630 | 1.67E−06 | GABA-B receptor 1 |
| 99667_at | NP_034073 | 2.36E−06 | Cytochrome c oxidase |
| | | | polypeptide VIa |
| | | | (EC 1.9.3.1) |
| 104343_f_at | NP_075685 | 2.36E−06 | Group XII-1 Phospholipase |
| | | | A2 |
| 99159_at | Q99KR7 | 5.58E−06 | Peptidyl-prolyl cis-trans |
| | | | isomerase, mitochondrial |
| | | | precursor (EC 5.2.1.8) |
| 97405_at | NP_033123 | 1.04E−05 | Ribosomal protein S6 |
| | | | kinase alpha 1 (EC 2.7.1.-) |
| 96212_at | AI853918 | 1.18E−05 | EST |
| 99934_at | NP_033016 | 2.46E−04 | Nectin-2; Poliovirus |
| | | | receptor related |
| | | | protein 2 |
| 96243_f_at | NP_064377 | 2.80E−04 | Aldehyde dehydrogenase |
| | | | 9A |
| 102123_at | NP_067435 | 3.60E−04 | Lysosomal acid lipase 1, |
| | | | Lip1 (EC 3.1.1.13) |
| 96296_at | NM_014175 | 4.64E−04 | Mrpl15: mitochondrial |
| | | | ribosomal protein L15 |
| 102240_at | NP_032930 | 3.01E−03 | PGC1; Peroxisome |
| | | | proliferative activated |
| | | | receptor, gamma, |
| | | | coactivator 1 |
| 94369_at | NP_062298 | 8.44E−02 | Glucosamine-phosphate N- |
| | | | acetyltransferase 1 |
| 97893_at | NP_035733 | 5.54E−01 | TATA box binding protein- |
| | | | like protein; TBP-like |
| | | | protein; TBP-like |
| | | | factor |

TABLE II
Identity of genes that are down-
regulated in response to PPARγ ligands

| AffyID | Genbank | P value | GeneName |
|---|---|---|---|
| 97926_s_at | NP_035276 | 6.84E−16 | Peroxisome proliferator |
| | | | activated receptor gamma |
| | | | (PPAR-gamma). |
| 92851_at | NP_031778 | 2.81E−15 | Ceruloplasmin (EC |
| | | | 1.16.3.1) |
| 97317_at | NP_056559 | 1.09E−13 | Phosphodiesterase |
| | | | I/nucleotide |
| | | | pyrophosphatase 2 |
| 103029_at | NP_035180 | 1.09E−13 | TIS; Topoisomerase |
| | | | inhibitor suppressed |
| 102395_at | NP_032911 | 2.84E−13 | Peripheral myelin protein |
| | | | 22 (PMP-22) (Growth- |
| | | | arrest-specific protein 3) |
| 100530_at | NP_033084 | 3.84E−13 | Ral guanine nucleotide |
| | | | dissociation stimulator |
| | | | (RaIGDS). |
| 96092_at | NP_059066 | 4.47E−13 | Haptoglobin |
| 97529_at | NP_038501 | 4.83E−13 | Annexin A8 |
| 103033_at | P01029 | 6.08E−13 | Complement C4 precursor |
| 104761_at | AA612450 | 7.11E−13 | EST |
| 98467_at | NP_061216 | 2.76E−12 | Inter alpha-trypsin inhibitor, |
| | | | heavy chain 4 (PK-120 |
| | | | precursor) |
| 92537_g_at | NP_038490 | 3.83E−12 | Beta-3 adrenergic |
| | | | receptor. |
| 103254_at | AW049897 | 3.83E−12 | EST |
| 100436_at | NP_032794 | 8.15E−12 | Alpha-1-acid glycoprotein |
| | | | 1 (AGP 1) (Orosomucoid |
| | | | 1) |
| 93354_at | NP_031495 | 8.87E−12 | Apolipoprotein C-I (Apo- |
| | | | CI). |
| 160319_at | NP_034227 | 1.05E−11 | SPL1; Extracellular matrix |
| | | | protein 2 |
| 94449_at | AI854522 | 1.93E−11 | Protocadherin 13 |
| 93543_f_at | NP_032209 | 3.28E−11 | Glutathione S-transferase |
| | | | Mu 2 (EC 2.5.1.18) |
| 93750_at | NP_034484 | 4.29E−11 | Gelsolin (Actin- |
| | | | depolymerizing factor) |
| | | | (Brevin) |
| 101973_at | NP_034958 | 4.70E−11 | CBP/p300-interacting |
| | | | transactivator 2 (MRG1 |
| | | | protein). |
| 97456_at | AI838021 | 7.40E−11 | Fatty acid CoA ligase, long |
| | | | chain 5 |
| 160306_at | NP_033407 | 1.07E−10 | SPOT14 (Thyroid |
| | | | hormone-inducible hepatic |
| | | | protein) |
| 100069_at | NP_031843 | 2.07E−10 | Cytochrome P450 2F2 |
| | | | (Naphthalene |
| | | | dehydrogenase) (EC |
| | | | 1.14.14.) |
| 93290_at | NP_038660 | 2.50E−10 | Purine nucleoside |
| | | | phosphorylase (EC |
| | | | 2.4.2.1) |
| 93090_at | NP_034337 | 4.92E−10 | BEK;Fibroblast growth |
| | | | factor receptor 2 (EC |
| | | | 2.7.1.112) |
| 97844_at | NP_033087 | 4.92E−10 | Regulator of G-protein |
| | | | signaling 2 (RGS2). |
| 160253_at | AW125390 | 2.71E−09 | IFITM3; interferon induced |
| | | | transmembraneprotein 3 |
| 93264_at | Q9WTN3 | 2.71E−09 | Sterol regulatory element |
| | | | binding protein-1 (SREBP- |
| | | | 1) |
| 97473_at | NP_444312 | 5.61E−09 | Transmembrane 4 |
| | | | superfamily, member 7 |
| 97426_at | NP_034258 | 7.71E−09 | Epithelial membrane |
| | | | protein-1 (EMP-1) |
| 102255_at | NP_035149 | 1.63E−08 | Oncostatin receptor |
| 93009_at | NP_032209 | 1.63E−08 | Glutathione S-transferase |
| | | | Mu 2 (EC 2.5.1.18) |
| 97950_at | NP_035853 | 9.70E−08 | Xanthine |
| | | | dehydrogenase/oxidase |
| | | | (EC 1.1.1.204) |
| 102366_at | NP_075360 | 9.70E−08 | Resistin |
| 98575_at | P19096 | 1.93E−07 | Fatty acid synthase (EC |
| | | | 2.3.1.85) |
| 100154_at | Q9R233 | 2.44E−07 | TAP-binding protein (TAP- |
| | | | associated protein). |
| 99577_at | NP_038626 | 3.08E−07 | Kit ligand; Mast cell growth |
| | | | factor |
| 97803_at | NP_032647 | 4.38E−07 | MPP1; 55 kDa erythrocyte |
| | | | membrane protein (P55) |
| 104153_at | NP_062800 | 4.94E−07 | IVD; Isovaleryl coenzyme |
| | | | A dehydrogenase |
| 160954_at | NP_038709 | 1.29E−06 | Synapsin II |
| 94057_g_at | NP_033153 | 3.01E−06 | Stearoyl-CoA desaturase 1 |
| | | | (Acyl-CoA desaturase 1) |
| | | | (EC 1.14.99.5) |
| 101058_at | NP_031472 | 1.91E−04 | Amylase 1 |
| 93496_at | AI852098 | 4.33E−03 | Fatty acid elongase 1 |
| 102689_at | AF110520 | 4.61E−01 | EST |

TABLE III
Identities of genes that are up-regulated by PPARγ ligands
as determined by Z score.

| AffyID | Genbank | p value | GeneName |
|---|---|---|---|
| 101979_at | NP_035947 | 4.26E−16 | Growth arrest and DNA- |
| | | | damage-inducible protein |
| | | | GADD45 gamma |
| 104325_at | gb\|AI461631 | 2.84E−13 | EST |
| 98132_at | NP_031834 | 4.47E−13 | Cytochrome c, somatic |
| 161042_at | gb\|AI324801 | 7.48E−12 | EST |
| 96879_at | Q60597 | 8.87E−12 | 2-oxoglutarate dehydrogenase |
| | | | E1 component (EC |
| | | | 1.2.4.2) |
| 160807_at | NP_443747 | 1.25E−11 | 1-acylglycerol-3-phosphate |
| | | | O-acyltransferase 3 |
| 102004_at | gb\|AI530403 | 6.17E−11 | Peroxisomal 3-oxoacyl |
| | | | CoA thiolase (ACAA1) |
| 95066_at | Q93092 | 4.92E−10 | Transaldolase (EC 2.2.1.2). |
| 103888_at | Q9WVB0 | 4.92E−10 | RNA-binding protein with |
| | | | multiple splicing (RBP- |
| | | | MS) |
| 97284_at | gb\|AI853789 | 1.33E−09 | EST |
| 92615_at | AI853615 | 1.33E−09 | EST |
| 92592_at | NP_034401 | 1.80E−09 | Glycerol-3-phosphate |
| | | | dehydrogenase [NAD+] (EC |
| | | | 1.1.1.8) |
| 97515_at | NP_032318 | 3.33E−09 | Estradiol 17 beta- |
| | | | dehydrogenase |
| | | | 4 (EC 1.1.1.62) |
| 160345_at | NP_444392 | 1.18E−08 | 60S ribosomal protein |
| | | | L34, mitochondrial |
| | | | precursor |
| 99666_at | gb\|AW12531 | 2.44E−07 | Citrate synthase |
| 104057_at | NP_077798 | 3.08E−07 | GrpE-like 1, mitochondrial |
| 94778_at | NP_036051 | 3.08E−07 | Aldehyde dehydrogenase1 |
| 160373_i_at | AI839175 | 7.05E−07 | Phosphatidylserine-binding |
| | | | protein |
| 160481_at | NP_035174 | 7.94E−07 | Phosphoenolpyruvate |
| | | | carboxykinase [GTP] (EC |
| | | | 4.1.1.32) (PEPCK) |
| 104100_at | NP_033012 | 7.16E−06 | Polymerase I and |
| | | | transcript release factor |
| 97871_at | NP_056589 | 1.18E−05 | ERO1-like |
| 160101_at | NP_034572 | 1.18E−05 | Heme oxygenase 1 |
| | | | (EC 1.14.99.3) |
| 160568_at | NP_075608 | 2.51E−05 | Alpha enolase (EC 4.2.1.11) |

TABLE IV
Identities of genes that are down-regulated by PPARγ ligands
as determined by Z score.

| AffyID | Genbank | p value | GeneName |
|---|---|---|---|
| 103556_at | AI840158 | 6.08E−13 | EST |
| 97885_at | NP_075543 | 2.34E−12 | LR8 protein |
| 104714_at | AW125299 | 8.87E−12 | EST |
| 97867_at | NP_032314 | 1.77E−11 | Corticosteroid 11-beta- |
| | | | dehydrogenase-1 (EC |
| | | | 1.1.1.146) |
| 99979_at | NP_034124 | 2.30E−11 | Cytochrome P450 1B1 |
| | | | (EC 1.14.14.1) (CYPIB1) |
| 101912_at | AI019679 | 3.28E−11 | EST |
| 92567_at | NP_031763 | 3.28E−11 | Procollagen, type V, alpha 2 |
| 93497_at | P01027 | 1.88E−10 | Complement C3 precursor |
| 99051_at | P07091 | 4.92E−10 | Placental calcium-binding |
| | | | protein (18A2) (PEL98) |
| 160255_at | AA657044 | 4.92E−10 | Neuroblast differentiation |
| | | | associated protein |
| 101123_at | NP_032436 | 4.92E−10 | Integral membrane |
| | | | protein 2B (E25B |
| | | | protein). |
| 98472_at | Y00629 | 4.92E−10 | Histocompatibility 2, |
| | | | T region locus |
| 102327_at | NP_033805 | 2.44E−09 | Vascular adhesion protein-1 |
| | | | (VAP-1) (EC 1.4.3.6) |
| 102094_f_at | AI841270 | 6.23E−09 | EST |
| 99024_at | NP_034883 | 6.23E−09 | MAX-interacting |
| | | | transcriptional |
| | | | repressor MAD4. |
| 93534_at | NP_031859 | 3.16E−08 | Decorin (PG-S2) |
| 97160_at | NP_033268 | 9.70E−08 | SPARC precursor |
| | | | (Osteonectin) (ON) |
| 96346_at | NP_149026 | 9.70E−08 | Cysteine dioxygenase 1 |
| 98331_at | P08121 | 9.70E−08 | Collagen alpha 1(III) |
| | | | chain |
| 92770_at | NP_035443 | 1.53E−07 | Calcyclin (Prolactin |
| | | | receptor associated protein) |
| 93077_s_at | NP_034871 | 1.53E−07 | Lymphocyte antigen |
| | | | Ly-6C precursor. |
| 93078_at | NP_034868 | 2.44E−07 | Lymphocyte antigen |
| | | | (T-cell-activating protein) |
| | | | (TAP). |
| 93100_at | NP_033738 | 3.01E−06 | Actin, alpha, cardiac |
| 102108_f_at | AI505453 | 8.72E−04 | EST |

### Example 2

### PPARγ1/2 Expression Measured in 3T3-L1 adipocytes treated with a PPARγ ligand

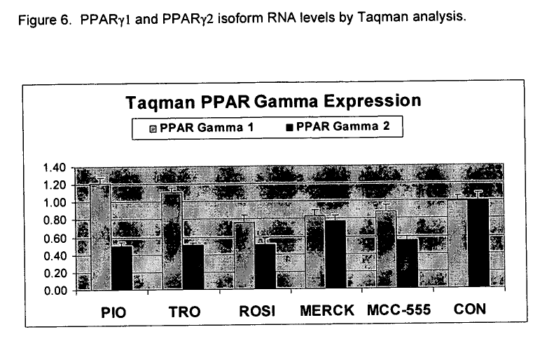

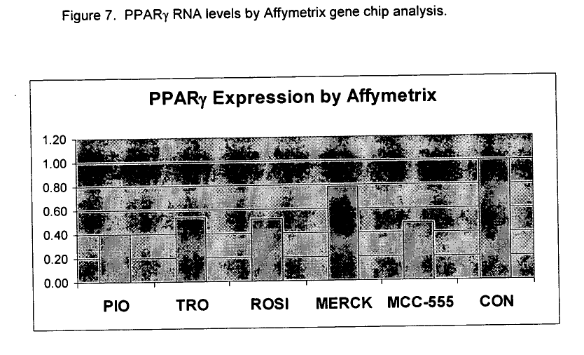

The expression levels of the PPARγ1 and PPARγ2 isoforms were measured by Taqman in the 3T3-L1 adipocyte cells in the presence and absence of PPARγ ligands. FIG. 6 shows the PPARγ1 and PPARγ2 expression levels in the presence of one of the 4 TZD ligands Troglitazone (Tro) 20 μM (Pfizer), Pioglitazone (Pio) 20 μM (Pfizer), MCC-555 20 μM (Pfizer), Rosiglitazone (Rosi) 1 μM (Pfizer), or the non-TZD PPARγ partial agonist/antagonist 5-chloro-1-(4-chlorobenzyl)-3-(phenylthio)-1H-indole-2-carboxylic acid (SPPARM) 20 μM (Pfizer) relative to a control treated sample (vehicle treated, 0.1% DMSO). These results show that both PPARγ isoforms are expressed in the adipocytes in the presence of ligand, and PPARγ2 expression is selectively down-regulated by treatment with PPARγ ligands while PPARγ1 expression level is largely unchanged.

The expression levels of PPARγ in 3T3-L1 adipocytes was also measured using Affymetrix microarray analysis. Following the incubation, RNA was extracted from the cells using Trizol (Invitrogen) followed by cleanup using an RNeasy kit (Qiagen) including DNase I treatment. Double stranded cDNA was synthesized with the SuperScript Choice system (Invitrogen) using a T7-(dT)₂₄ oligomer (Ambion) from the resulting RNA from each of these cell populations. Biotin labeled probes from the cDNAs were subsequently obtained by in vitro transcription using the BioArray High Yield RNA Transcript Labeling Kit (Enzo). The probes were then hybridized to U74Aver2 Affymetrix gene chips. The chips were hybridized for 16 hours at 45° C. and 60 RPM in a rotisserie box. The results, which are shown in FIG. 6 confirms that treatment of adipocytes with PPARγ ligands results in down-regulation of PPARγ expression.

### Example 3

### Correlation of “on-PPARγ Genes/Off-PPARγ Genes” Ratio with Edema Frequency

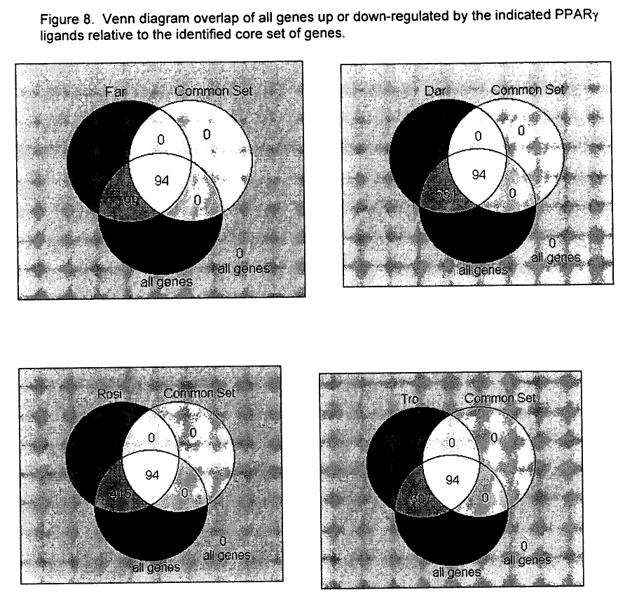

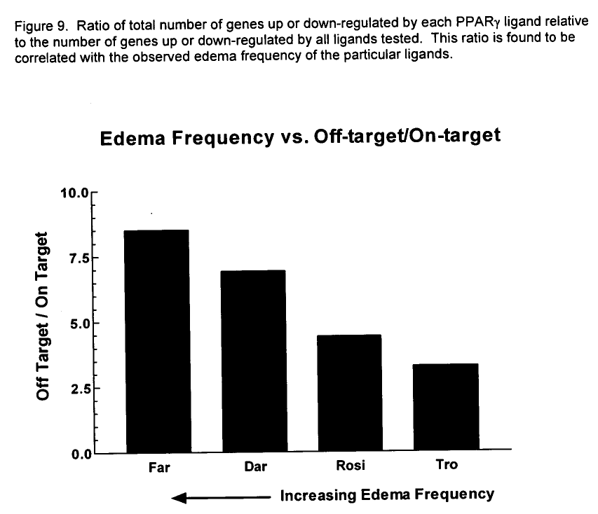

This example shows that, the ratio of the total number of genes that are up-regulated for a particular ligand relative to the number of genes that are up-regulated by all six ligands tested correlates with the frequency of edema observed in patients. All of the PPARγ ligands tested cause edema clinically with differing frequency and severity. Farglitazar, the most potent PPARγ ligand reported, has the most frequent incidence of edema clinically followed by Darglitazone, Rosiglitazone and Troglitazone respectively (Heidi Camp, Pfizer Inc, personal communication). We have observed that each PPARγ ligand tested, up or down-regulates an independent set of genes in addition to the set of genes up or down-regulated by all ligands. We find that the ligands that modulate expression of a larger number of genes outside the common set of genes, are the ligands that have the greater frequency of clinical edema. Similarly, the ligands that modulate a smaller set of genes relative to the common set of genes for all ligands, are those ligands with lower observed frequency of edema. The overlapping set of genes modulated by all PPARγ ligands is considered “on target” and important for insulin sensitizing efficacy, while the non-overlapping set is termed “off target” and may reflect genes responsible for the side effect profiles of the individual agents. We hypothesize that an ideal insulin sensitizing PPARγ ligand will modulate only this core set of on target genes. The results as Venn overlap indicating the number of genes modulated for each ligand relative to the common gene set, and the off target/on target ratios for each ligand relative to edema frequency are shown in FIGS. 8 and 9.

### Example 4

### Gene Profile of 3T3-L1 Cells Treated with Rosiglitazone after PPARγ1 Expression Knockdown

3T3-L1 cells were treated for 72 hours with an antisense construct whose nucleic acid sequence was complementary to PPARγ1 and treated with Rosiglitazone. As a separate control, 3T3-L1 were cells treated with the antisense construct, but without Rosiglitazone. Gene expression levels were determined using Affymetix microarray analysis. Following the incubation, RNA was extracted from the cells using Trizol (Invitrogen) followed by cleanup using an RNeasy kit (Qiagen) including DNase I treatment. Probes from each of these cell populations were prepared by reverse transcription-polymerase chain reaction (RT-PCR) and subsequently biotin labeled by in vitro transcription using the BioArray High Yield RNA Transcript Labeling Kit (Enzo). The probes were then hybridized to U74Aver2 Affymetrix gene chips. The hybridization was conducted as follows The hybridization was for 16 hours at 45° C. and 60 RPM in a rotisserie box. The results indicated that 91% of the genes that were found to be up- or down-regulated in response to a PPARγ ligand, as described in Example 1 (Tables I and II), were also found to be up- or down-regulated by Rosiglitazone treatment in cells treated with the PPARγ1 antisense nucleic acid. However, the remaining genes were no longer up- or down-regulated in response to Rosiglitazone in cells treated with the PPARγ1 antisense nucleic acid. These genes are listed in Table V.

These results indicate that the expression signature obtained for the PPARγ ligands may contain a subset of genes that are PPARγ1 isoform specific and a subset that are PPARγ2 specific. Furthermore, PPARγ2 appears to be the isoform primarily responsible for the expression profile obtained in mature 3T3-L1 adipocytes.

TABLE V
Identities of genes no longer regulated by Rosiglitazone treatment
after PPARγ1 antisense knockdown.

| Probe | Description |
|---|---|
| 97950_at | Xanthine dehydrogenase |
| 100436_at | AGP1 |
| 93290_at | Purine nucleoside Phosphorylase |
| 104746_at | FK506 binding protein |
| 99667_at | Cytochrome c oxidase |

### Example 5

### Confirmation of Core Gene Set with Independently Performed Experiment

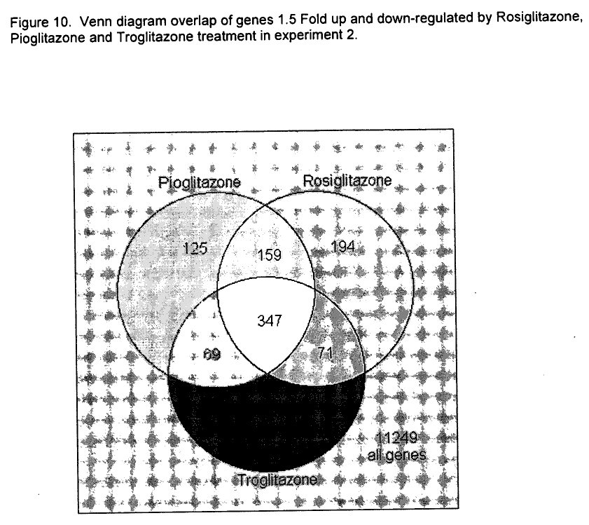

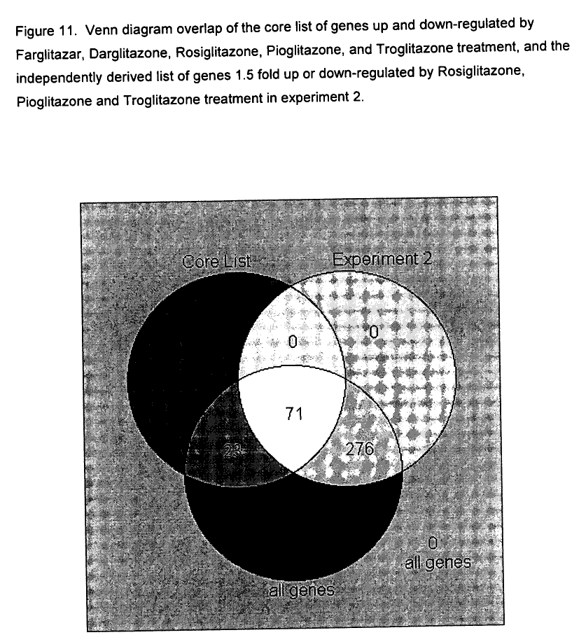

The common set of genes that were determined to be 1.5 fold up or down-regulated in 3T3-L1 adipocytes by all PPARγ ligands tested as described in Example 1 (Tables I and II) were independently confirmed by microarray analysis. In this experiment, 3T3-L1 adipocytes were treated for 24 hours with either 1 μM Rosiglitazone (Avandia, GlaxoSmithKline), 20 μM Pioglitazone (Actos, Takeda Pharmaceuticals), Troglitazone (Rezulin, Parke-Davis), or vehicle (0.1% DMSO). RNA was isolated with Trizol (Invitrogen) followed by DNase I treatment and cleanup with RNeasy kit (Qiagen). Double stranded cDNA was synthesized with the SuperScript Choice system (Invitrogen) using a T7-(dT)₂₄ oligomer (Ambion) from the resulting RNA from each of these cell populations. Biotin labeled probes from the cDNAs were subsequently obtained by in vitro transcription using the BioArray High Yield RNA Transcript Labeling Kit (Enzo). The probes were then hybridized to the older version U74A Affymetrix gene chips. The hybridization was for 16 hours at 45° C. and 60 RPM in a rotisserie box. Using GeneSpring (Slilicon Genetics) software, genes that are up or down-regulated by 1.5 fold or greater for all three PPARγ ligands were determined as shown by Venn diagram overlap in FIG. 10. This gene list was then compared to the common set of genes determined previously to be up or down regulated 1.5 fold or greater by Farglitazar, Darglitazone, Rosiglitazone, Troglitazone, and Pioglitazone. As shown by Venn diagram overlap in FIG. 11, of the 94 genes modulated by all 5 PPARγ ligands in the first experiment, 71 were confirmed by the second experiment. Further analysis revealed that probe sets were not present on the older U74A gene chips for 8 genes out of the 23 that were not confirmed by the second experiment. Additionally, 10 of the remaining 15 genes that had probes on both versions of chips but were not confirmed in the second experiment, were found to be up or down-regulated by at least 2 of the 3 PPARγ ligands tested. This example provides confirmation of the core set of genes that are up or down-regulated by all five PPARγ ligands.

We claim:

1. A method for identifying a therapeutic having analogous activity to a thiazolidinedione comprising contacting a cell containing a PPARγ receptor with a candidate therapeutic; and determining the level of expression of at least one gene selected from the panel of genes in Table I and/or Table II, wherein an increase in the level of expression of at least one gene of Tables I or III and/or a decrease in the level of expression of at least one gene of Tables II or IV in the cell treated with the candidate therapeutic relative to a cell that was not treated with the candidate therapeutic indicates that the candidate therapeutic is a therapeutic for treating a disease associated with a PPARγ receptor.

2. The method of claim 1, wherein said candidate therapeutic is selected from the group consisting of: proteins, peptides, peptidomimetics, derivatives of fafty acids, and small molecules.

3. The method of claim 1, wherein said disease is Type II diabetes.

4. The method of claim 1, wherein said disease is obesity.

5. The method of claim 1, wherein said disease is treatable by a thiazolidinedione.

6. The method of claim 1, wherein said PPARγ receptor is the PPARγ1 receptor.

7. The method of claim 1, wherein said PPARγ receptor is the PPARγ2 receptor.

8. The method of claim 1, wherein said candidate therapeutic is in a library of compounds.

9. The method of claim 1, wherein the expression level of at least three genes is detected.

10. The method of claim 1, wherein the expression level of at least ten genes is detected.

11. A composition comprising a plurality of genes or gene fragments selected from the panel of genes in Tables I-IV.

12. The composition of claim 11, wherein the plurality is at least 10 genes or gene fragments.

13. The composition of claim 12, wherein the plurality is at least 20 genes or gene fragments.

14. The composition of claim 12, which is a chip, wafer or slide.

15. A composition comprising a plurality of proteins or proteins fragments selected from proteins encoded by the panel of genes in Tables I-IV.

16. The composition of claim 15, wherein the plurality is at least 10 proteins or protein fragments.

17. The composition of claim 16, wherein the plurality is at least 20 proteins or protein fragments.

18. The composition of claim 15, which is a chip, wafer or slide.

19. A method for determining whether a subject is responsive to treatment with a therapeutic having analogous activity to a thiazolidinedione, comprising determining the level of expression of a plurality of genes of Tables I or III or Tables II or IV in cells of the subject, wherein a higher level of expression of the genes of Tables I or III or a lower level of expression of the genes of Tables II or IV in the adipocytes of the subject relative to that in adipocytes of a subject that was not treated with a PPARγ ligand indicates that the subject is responsive to treatment with the PPARγ ligand.

20. A method of claim 19, wherein the cells are adipocytes.

21. A method for predicting whether a subject would be responsive to treatment with a compound having analogous activity to a thiazolidinedione, comprising incubating cells of the subject with a PPARγ ligand and determining the level of expression of a plurality of genes of Tables I and/or III and/or Tables II and/or IV in the cells, wherein a higher level of expression of genes of Tables I or III or lower level of expression of genes of Tables II or IV relative to expression in cells of subjects not treated with a PPARγ ligand indicates that the subject would be responsive to treatment with the PPARγ ligand.
